(19) United States
(12) Patent Application Publication
(10) Pub. No.: US 2004/0241152 A1
(43) Pub. Date: Dec. 2, 2004

(54) Methods for inducing an immune response with an elevated th1/th2 ratio, by intracellular induction of nfkappab

(76) Inventors: Brian Maurice John Foxwell, London (GB); Marc Feldmann, London (GB)

Correspondence Address: NIXON & VANDERHYE, PC, 1100 N GLEBE ROAD, 8TH FLOOR, ARLINGTON, VA, 22201-4714, US

(21) Appl. No.: 10/482,851

(22) PCT Filed: Jul. 5, 2002
(86) PCT No.: PCT/GB02/03155

(30) Foreign Application Priority Data
Jul. 5, 2001 (GB) 0116249.4

(51) Int. Cl. A61K 45/00

(52) U.S. Cl. 424/93.71

(57) ABSTRACT

The present invention provides a method of increasing the T_{H1}:T_{H2} ratio of an immune response, containing the step of supplying to an antigen presenting cell (APC) such as a dendritic cell (DC) or precursor cell, an intracellular activator of APC, such as DC, function. The invention also provides a method of treating a patient with or at risk of allergy comprising the step of supplying an intracellular activator of APC, such as DC, function, or an intracellular inducer of NF_{κ}B, to the patient or to an APC, such as a DC, or precursor cell, of the patient.

The present invention relates to modulation of the immune system, particularly modulation of response to allergens.

Antigen presentation is a critical step in the initiation of the immune response. Antigen presenting cells are well known in the art and include dendritic cells (see Janeway, C A Jr & Tavers, P, Immunobiology (3rd Edition), Editions Current Biology/Churchill Livingstone and Garland Publishing). They are highly specialised cells that can process antigens and display their peptide fragments on the, cell surface, together with molecules required for lymphocyte activation. The most potent antigen-presenting cells are dendritic cells, macrophages and B cells. Dendritic cells (DC) are considered to be the most potent antigen presenting cells for naive T cells. This is partly due to their high expression of MHC and costimulatory molecules (Hart (1997) Blood 90, 3245-3287). However, little is known about the biochemical pathways which regulate antigen presenting function, partly due to the difficulty in transfecting DC.

Dendritic cells are bone marrow derived cells which were first described in the early 1970's by Steinman and Cohn (1973) J. Exp. Med 179, 1109. Studies on dendritic cells were initially hampered by tie difficult in isolating them in sufficient numbers, but his problem was overcome in part by the realisation that a subset of DC could be generated in vitro by culture of CD34+ cells or human monocytes with GM-CSF and IL-4. These cultured DC have the phenotype of immature DC, and can be matured into high MHC, high CD80/96 expressing cells through incubation with TNFα or LPS (Bender et al (1996) J. Immunol. Methods 196, 121; Romani et as (1996) J. Immunol. Methods (1996) 196, 137; Reddy et al (1997) Blood 90, 3640).

DC can also be derived from a post colony-forming unit CD14⁺ intermediate in the peripheral blood. DC migrate to peripheral sites in skin, mucosa, spleen and thymus. They have been implicated in a variety of clinically important processes, including allograft rejection, atopic disorders, autoimmunity and anti-tumour immunity.

DC can be cultured ex vivo from CD34⁺ stem cells or CD14⁺ peripheral blood monocytes using cytokines, principally GM-CSF, IL-4 and TNFα Scabolsc et al (1995) J. Immunol. 154, 5651-5661. DC from both these sources are immunocompetent and can take up exogenously presented antigen, process it and then present it to cytotoxic T-cells (Grabbe et al (1995) Immunology Today 16, 117-121; Girolomoni & Ricciardi-Castagnoli (1997) Immunology Today 18, 102-104). DC can transfer antigen-specific tumour immunity generated in vivo (Kwak et al (1995) Lancet 345, 1016-1020) and autologous DC pulsed with tumour antigen ex vivo can induce a measurable anti-tumour effect (Hsu et al (1996) Nature Medicine 2, 52-58). DC can be effectively pulsed using a crude tumour membrane lysate, purified peptides or peptide fragments. The ex vivo expansion of autologous dendritic cells from patients, loading with a peptide antigen and reinfusion as adoptive immunotherapy, is described in, for example, WO/00/26249.

The importance of antigen presentation in the generation of immune response was confirmed by demonstration that blocking antigen presentation downregulates immune responses and is useful in treating animal models of disease. Thus antibody to murine MHC class II has been used to treat experimental allergic encephalomyelitis (Smith et al (1994) Immunology 83, 1), and blocking the CD80/86 costimulatory molecules with antibodies or CTLA4-Ig fusion protein is beneficial in transplants or animal models of arthritis (Lu et al (1999) Gene Ther. 6, 554-563). This has led to a search of new ways of downregulating antigen presentation which may be useful in human diseases or in transplantation.

Allergic diseases such as asthma, atopic dermatitis and hayfever are driven in large part by T_{H2} cytokine dependent antibody responses. The most critical T_{H2} cytokines are IL-4 and IL-5, and the most important antibody response is IgE.

IgG2a antibody levels correlate with T_{H1} and IgG1 antibody levels with T_{H2} profiles (Mosmann T. R. and Coffman R. L. 1989).

The therapy of allergic disease is currently chiefly symptomatic, with corticosteroids most widely used. However, this has no impact on the underlying abnormal immune response or its cause. There is therefore a need for further methods for treating patients with or at risk of allergy.

We have surprisingly shown that activators of antigen presenting cell, for example dendritic cell, function, for example inducers of NF-κB are useful in increasing the T_{H1}:T_{H2} ratio of an immune response and in treating allergy.

NF-κB has been speculated as being involved in the immune system. This is summarised in, for example, the paper by Baeueurle P. A. and Henkel T. (Annual Reviews in Immunology, 1994, Vol. 12, pages 141-179). The activation of the transcription factor NF-κB like proteins results from post-translational modification permitting translocation of the preformed transcription factor from the cytoplasm to the nucleus. This translocation is controlled by the phosporylation and degradation of an inhibitor protein called IκB, which forms a complex with NF-κB, and thereby holds it in the cytoplasm. Stimulation of the cell by appropriate signals leads to modification of IκB which in turn results in its dissociation and/or degradation from NF-κB.

Binding of the IκB protein to NF-κB masks the nuclear localisation signal (NLS) of NF-κB. Upon stimulation of the cell with specific agents, which depend on the cell type and stage of cell development, IκB is modified in a way that disables binding to NF-κB, leading to dissociation of NF-κB from IκB.

NF-κB is a heterodimeric protein consisting of a 50 kD subunit (p50) and a 65 kD subunit (p65). The cDNAs for p50 and p65 have been cloned and have been shown to be homologous over a region of 300 amino acids.

An additional member of the NF-κB family, Rel B, has been cloned as an immediate early response gene from serum-stimulated fibroblasts.

Both p50 and p65 are capable of forming homodimers, although with different properties: whereas p50 homodimers have strong DNA binders affinity but cannot transactivate transcription, the p65 homodimers can only weakly bind to DNA but are capable of transactivation. P50 is synthesised as the amino-terminal part of the 110 kD precursor (p110), which has no DNA binding and dimerisation activity. The carboxyl-terminal part contains eight ankyrin repeats, a motif found in several proteins involved in cell cycle control and differentiation.

Five IκB family members have been identified: IκBα, IκBβ, p105/IκBγ, p110/IκBΔ and IκBε (Baeuerle and Baltimore, Cell 1996, Vol. 87, pages 13-20). All IκB-like family members contain multiple ankyrin repeats, which are essential for inhibition of NE-κB activation.

PCT/GB00/04925 concerns activation and inhibition of the immune system using intracellular activators or inhibitors of APC function, for example using inducers or inhibitors of NFκB.

Central to the recognition mechanisms of the immune system are a number of germline-encoded receptors known as toll-like receptors (TLRs) (1). Individual TLRs activate specialised anti-fungal or anti-bacterial genes through the activation of the NF-κB transcription factors (2). Thus, TLR4 has been shown to confer responsiveness to bacterial lipopolysaccharide (3) whereas TLR2 confers responsiveness to bacterial peptidoglycan and lipoteichoic acid as well as yeast carbohydrates (4). 9 TLRs are currently known (8) and many more expected to exist.

Although the extracellular portions of Toll-related receptors (TRRs), including TLRs, are relatively divergent, the cytoplasmic portions are more conserved. They contain a well-defined region known as the toll domain, which is also found in the cytoplasmic portion of proteins comprising the IL-1 receptor, the IL-18 receptor and other receptors broadly termed the IL-1 receptor family. In addition, soluble cytoplasmic proteins such as MyD88 can have Toll domains. TLRs and IL-1 receptor use an analogous framework of signalling; upon ligand binding, they recruit the adaptor molecule MyD88 through homotypic interactions with a toll domain that MyD88 contains in its C-terminus. MyD88, in turn, recruits IRAK, TRAF-6 and Tol1IP to activate NF-κB and mitogen-activated protein kinases (2; Burns et al (2000) Nature cell Biol. 2, 346-351).

The MyD88 (myeloid differentiation protein) is considered to have a modular organisation consisting of an N-terminal death domain (DD) separated by a short linker from a C-terminal Toll domain (reviewed in (5)). The N-terminal DD is related to a motif of approximately 90 amino acids that is considered to mediate protein-protein interactions with other DD sequences forming either homo- or heterodimers (Boldin et al (1995) J Biol Chem 270, 387-391).

The MyD88 Toll domain has about 130 amino acids (Mitcham et al (1996) J Biol Chem 199, 144-146). Toll domains are also considered to mediate protein-protein interactions with other Toll domains forming either homo- or heterodimers (see (5)).

DD and Toll-Toll interactions are considered to be involved in directing signalling pathways. MyD88 is considered to bind via its Toll domain to TLRs and the IL-1 receptor (when bound to ligand). In turn, MyD88 is considered to bind via its DD to other DD-containing proteins; in particular it is considered to bind to IRAK and TRAF-6, thereby activating NF-κB and mitogen-activated protein kinases (2).

We have previously shown that there also is an inhibitory signal, specific for antigen presenting cells (APCs) such as dendritic cells and macrophages, that acts through MyD88, as described in UK patent application No 0031454.2, filed on 22 Dec. 2000. The inhibitory signal may involve one or more TRRs. TRRs include molecules such as TLRs, IL-1 receptor family members including IL-1 receptor and IL-18 receptor and cytoplasmic proteins such as MyD88. Molecules that block TRR signalling in APCs, such as dendritic cells, for example loss-of-function (inhibitory eg dominant negative) forms of MyD88 (termed MyD88dn), may be used as activators of APC, for example DC, function.

A first aspect of the invention provides a method for increasing the T_{H1}:T_{H2} ratio of an immune response, comprising the step of supplying to an antigen presenting cell (APC) such as a dendritic cell (DC), or precursor cell, an intracellular activator of APC, such as DC, function.

By “increasing the T_{H1}:T_{H2} ratio of an immune response” is included the meaning that the ratio of IgG2a antibody concentrations to IgG1 antibody concentrations for a chosen antigen is increased. These concentrations correlate with T_{H1} and with T_{H2} profiles (Mosmann T. R. and Coffman R. L. 1989). The concentration of IgG2a antibodies may increase and/or the concentration of IgG1 antibodies may decrease, as described in Example 1. Proliferation of lymph node cells, IFNγ production, IL4, IL5 and/or IgE levels, or levels of other cytokines, may also be used in assessing the T_{H1}:T_{H2} ratio. For example, to examine antigen-specific T cell responses, an ex vivo assay that measures the proliferation of lymph node cells animals (Alkan S. S. 1978) may be used. This assay is chiefly used for T_{H1} responses as it is dependent on T cell proliferation and IL-2 production. Lymph node cell cultures may also be used to measure T_{H1}/T_{H2} cytokine profiles, either by analysis of the cell supernatant or intracellular FACS staining. IFNγ production is indicative of a T_{H1} response, whilst IL4 production is indicative of a T_{H2} response.

Comparisons may be made between treated and untreated individuals, or, preferably, between the concentrations for an individual before and after treatment, as well known to those skilled in the art.

For example, it is preferred that one or more indicators of the T_{H1}:T_{H2} ratio (for example relative levels of IgG1 to IgG2a antibodies) indicate that the T_{H1}:T_{H2} ratio is at least 1.2:1, 1.5:1, 1.8:1, 2:1, 3:1, 5:1, 10:1, 20:1, 30:1, 50:1, 70:1 or 100:1.

It will be appreciated that different ratios may be achieved in different subjects using the same activator; for example the ratio achieved in a BALB/c mouse (which is predisposed to generate a T_{H2}-type response) may not be the same as that achieved in a human.

A second aspect of the invention provides a method of increasing the T_{H1}:T_{H2} ratio of an immune response in a mammal, such as a human, comprising the step of supplying an intracellular activator of APC, such as DC, function to the mammal or to an APC, such as a DC, or precursor cell, of the mammal.

The invention accordingly provides a method of treating a patient in need of an increase in the T_{H1}:T_{H2} ratio of an immune response comprising the step of supplying an intracellular activator of APC, such as DC, function to the patient or to an APC, such as a DC, or precursor cell, of the patient.

The patient may be a mammal, for example a human, with or at risk of allergy. The patient may be atopic or have a family history of allergy or atopy. Criteria by which a patient may be judged to have an allergic condition or to be atopic will be well known to those skilled in the art and may include measurement of IgE levels. For example, Williams et al (1994) Br J Dermatol 131, 406-416 sets out diagnostic criteria for atopic dermatitis.

Allergy to ingested substances can manifest itself in a wide range of symptoms affecting any organ in the body. Commonly it affects particularly the gastrointestinal tract, the skin, the lung, the nose and the central nervous system. Allergic reactions to ingested substances affecting these organs can manifest themselves as abdominal pain, abdominal bloating, disturbance of bowel function, vomiting, rashes, skin irritation, wheezing and shortness of breath, nasal running and nasal blockage, headache and behavioural changes. In addition in severe food allergic reactions, the cardiovascular and respiratory systems can be compromised giving anaphylactic shock and in some cases death.

It is also recognised that in certain chronic diseases, allergy to ingested substances is the probable cause of the disease in a proportion of patients. These diseases include susceptibility to anaphylactic shock, atopic dermatitis, chronic urticaria, asthma, allergic rhinitis, irritable bowel syndrome, migraine and hyperactivity in children. It is also possible that food allergy may be a factor in certain patients with inflammatory bowel disease (ulcerative colitis and Crohn's disease).

Allergy to inhaled substances can manifest itself as rhinitis, asthma or hayfever. The respiratory tract and/or eyes may be affected. For example, asthma can be provoked by inhalation of allergen in the clinical laboratory under controlled conditions. The response is characterised by an early asthmatic reaction (EAR) followed by a delayed-in-time late asthmatic reaction) (See Allergy and Allergic Diseases (1997), A. B. Kay (Ed.), Blackwell Science, pp 1113 to 1130). The EAR occurs within minutes of exposure to allergen, is maximal between 10 and 15 min and usually returns to near baseline by 1 hour. It is generally accepted that the EAR is dependent on the IgE-mediated release of mast cell-derived mediators such as histamine and leukotrienes. In contrast the LAR reaches a maximum at 6-9 hours and is believed to represent, at least in part, the inflammatory component of the asthmatic response and in this sense has served as a useful model of chronic asthma.

The late asthmatic response is typical of responses to allergic stimuli collectively known as late phase responses (LPR). LPR is seen particularly in the skin and the nose following intracutaneous or intranasal administration of allergens.

Allergy by skin contact may manifest itself as eczema or atopic dermatitis. Atopic dermatitis is an inflammatory skin disorder, affecting up to 10% of the paediatric population. It is characterised by extreme itching, a chronic relapsing course and specific distribution around the body. There is usually a family history of allergy and the condition starts in early infancy. Typical treatment regimes are to use simple emollients or topical corticosteroids. Long-term use of topical corticosteroids may have undesirable side effects, particularly in children. Contact allergens include latex, detergents or other ingredients of washing powders, animal dander and house dust mites.

Serum IgE levels may be measured by techniques well known to those skilled in the art, for example using the Pharmacia & Upjohn UniCAP Total IgE Test, and preferably also the Pharmacia & Upjohn UniCAP Specific IgE Test and/or skin prick tests to suspected allergens.

Accordingly, a further aspect of the invention provides a method of treating a patient with or at risk of allergy comprising the step of supplying an intracellular activator of APC, such as DC, function to the patient or to an APC, such as a DC, or precursor cell, of the patient.

The activator may be an inducer of NFκB function, as discussed further below. Inducers of NFκB are also described in PCT/GB00/04925.

Accordingly, the invention further provides a method of increasing the T_{H1}:T_{H2} ratio of an immune response, comprising the step of supplying to an antigen presenting cell (APC) such as a dendritic cell (DC), or precursor cell, an intracellular inducer of NFκB. The invention further provides a method of increasing the T_{H1}:T_{H2} ratio of an immune response in a mammal, such as a human, comprising administering a pharmaceutically-effective dose of an intracellular inducer of NFκB.

A further aspect of the invention provides a method of treating a patient in need of an increase in the T_{H1}:T_{H2} ratio of an immune response comprising the step of supplying an intracellular inducer of NFκB to the patient or to an APC, such as a DC, or precursor cell, of the patient.

A further aspect of the invention provides a method of treating a patient with or at risk of allergy comprising the step of supplying an intracellular inducer of NFκB to the patient or to an APC, such as a DC, or precursor cell, of the patient.

A further aspect of the invention provides the use of an intracellular activator of APC, such as DC, function in the manufacture of a medicament for treating a patient in need of an increase in the T_{H1}:T_{H2} ratio of an immune response.

A further aspect of the invention provides the use of an intracellular inducer of NFκB in the manufacture of a medicament for treating a patient in need of an increase in the T_{H1}:T_{H2} ratio of an immune response.

A further aspect of the invention provides the use of an intracellular activator of APC, such as DC, function in the manufacture of a medicament for treating a patient with or at risk of allergy. A further aspect of the invention provides the use of an intracellular inducer of NFκB in the manufacture of a medicament for treating a patient with or at risk of allergy.

It is preferred that the activator of APC, such as DC, function is an intracellular inducer of NFκB. It will be appreciated that an intracellular inducer of NFκB may be considered to be an activator of APC, such as DC, function, but this may not be essential.

The activator or inducer may be a dominant negative mutant of MyD88 (termed MyD88dn) or a polynucleotide encoding MyD88dn, for example MyD881pr or a polynucleotide encoding MyD881pr, as discussed further below.

Alternatively, the activator or inducer may be MyD88 or a polynucleotide encoding MyD88.

A further aspect of the invention provides a method of treating a patient with or at risk of allergy or in need of an increase in the T_{H1}:T_{H2} ratio of an immune response, comprising the step of supplying to the patient, or to an antigen presenting cell, such as a dendritic cell, or precursor cell, of the patient, a dominant negative mutant of MyD88 (MyD88dn).

A further aspect of the invention provides the use of a dominant negative mutant of MyD88 (MyD88dn), or polynucleotide encoding MyD88dn, in the manufacture of a medicament for treating a patient with or at risk of allergy or in need of an increase in T_{H1}:T_{H2} ratio of an immune response.

A still further aspect of the invention provides a method of treating a patient with or at risk of allergy or in need of an increase in the T_{H1}:T_{H2} ratio of an immune response, comprising the step of supplying to the patient, or (less preferably) to an antigen presenting cell, such as a dendritic cell, or precursor cell, of the patient, MyD88 (ie a molecule having the signalling activity of wild-type MyD88 (termed Myd88wt) as discussed further below).

A further aspect of the invention provides the use of MyD88, or polynucleotide encoding MyD88, in the manufacture of a medicament for treating a patient with or at risk of allergy or in need of an increase in the T_{H1}:T_{H2} ratio of an immune response.

It is considered that MyD88 and dominant negative mutants of MyD88 may act on different signalling pathways within APCs and may both have the effect of increasing the T_{H1}:T_{H2} ratio of the immune response. Alternatively, or in addition, Myd88wt may be acting as an activator of cells other than dendritic cells, for example fibroblasts, for example by acting as an inducer of NFκB in those cells. The DNA of a DNA vaccine (for example naked DNA or virally delivered DNA) may enter and be expressed in cell types including muscle cells, fibroblasts or DCs. With Myd88wt the activation of the immune response may occur via activation of infected fibroblasts expressing the antigen.

A dominant negative mutant of MyD88, for example MyD881pr (as well as other activators of APC function) may act as an inhibitor of a Toll-related receptor (TRR) signalling pathway found in APCs, such as dendritic cells, or a precursor thereof. In further preference, the activator of APC function inhibits a TRR signalling pathway, the inhibition of which induces activation of immature dendritic cells and/or enhancement of antigen-presenting function and may induce NF-κB nuclear translocation or the activation of MAP kinases. Thus, the TRR signalling pathway is considered to contribute to maintenance of immature APCs, such as dendritic cells, in the immature form, and to maintenance of NF-κB in an inactive form. Activation of the TRR signalling pathway may reduce the response of immature APCs, such as dendritic cells, to maturing factors, for example GM-CSF and IL4, ie may reduce the number of mature APCs, such as dendritic cells, formed, or may increase the time or dose of maturing factors needed for a given number of mature APCs, such as dendritic cells, to form. Activation of the TRR signalling pathway may reduce the ability of mature APCs, such as dendritic cells, to induce a MLR (mixed lymphocate reaction), a test of APC, such as dendritic cell, function well known to those skilled in the art. The APCs, such as dendritic cells, are incubated with allogeneic T cells and proliferation of the cells is measured, for example by measuring tritiated thymidine uptake after 6 days. For example, 10⁵ T cell may be plated with graded doses (for example from 50 to 10000 per well) of dendritic cells in a 96-well round-bottom microtiter plate.

Typically, the APC is a professional antigen-presenting cell such as a dendritic cell, mucosal cell, macrophage or B cell. MHC Class II molecules are found in professional APCs. Professional APCs are characterised by the presence of costimulatory molecules such as CD80 and CD86 as defined by Mellman et al (1998) Trends Cell Biol. 8, 231-237.

Typically, isolated precursor or dendritic cells which are activated express higher levels of HLA-DR, MHC Class I and CD80/86 compared to unactivated cells.

A list of DC surface markers regulated upon enhancement of antigen-presenting function is given in Banchereau et al (2000) Ann. Rev. Immunol. Dendritic cell surface markers include high CCR1, CCR5, CCR6 but low CCR7 chemokine receptors; high CD68; low levels of MHC Class I (HLA-A, B, C) and MHC Class II (HLA-DR, HLA-DQ and HLA-DP); low co-stimulatory molecules such as CD40, CD54, CD80, CD83 and CD86 and no DC-LAMP. Activated DC with increased antigen presentation have low CCR1, CCR5, CCR6; high CCR7; low CD68; high surface MHC Class I and II; high co-stimulatory molecules such as CD40, CD54, CD58, CD80, CD83, CD86; high DC-LAMP and high p55 fascin.

Examples of molecules which act as activators of APC, for example DC, function and which may be useful in the present invention are described in GB Application No 0031454.2, supra and in PCT/GB00/04925, filed on 22 Dec. 2000.

For the avoidance of doubt, cytokines and molecules containing a CpG motif are not intracellular inducers or enhancers of APC function since they act extracellularly.

It is preferred that the activator molecule leads to activation of NF-κB in the APC. For example, it may increase NF-κB activation/nuclear translocation and/or gene transactivation.

It is preferred that the intracellular inducer of NFκB induces NFκB in APCs, for example DCs. Alternatively or in addition, it may induce NFκB in other cell types, for example fibroblasts.

For example, MyD88dn (for example Myd881pr) may induce NFκB in APCs, whilst Myd88wt may induce NFκB in other cells, for example fibroblasts. They may also activate the MAPK kinase pathways (p38, p54/JNK, p42/44 Erk) in different cells.

Example of activators or inducers of NFκB include TRAFs (including. TRAFs 2, 3, 4, 5, 6,), TRADD, NIK, IKK1, IKK2, TAK, PKR, NAK, MEKK, p65/relA, c-rel and rel B. Other activators or inducers include p38MAK, p54JNK, p42/44Erk, MEKs (1, 2, 3, 4, 5, 6, 7,) or MEKKs, for example wild-type or activated mutants of any of these kinases.

By “intracellular activator of APC function” we include any suitable activator of antigen presenting cell function. By “APC function” we include the ability to present antigen, the ability to express MHC Class II, the ability to express cell surface molecules such as costimulatory molecules including CD80 and CD86, the ability to produce cytokines and the ability to induce activation rather than anergy. Typically the activator of APC function is an activator of DC function. Preferably, the activator is an activator of intracellular signalling within the APC. By “intracellular signalling within the APC” we include communication between the membrane and the nucleus, signalling winch controls gene expression (including expression of CD80 and CD86) and control of cytoskeletal organisation. Activators of intracellular signalling include, for example, an inducer of NF-κB as described in more detail below.

Antigen presentation describes the display of antigen as peptide fragments bound to MHC molecules on the surface of a cell; T cells recognise antigen only when it is presented in this way.

By pharmaceutically-effective dose, we mean an amount sufficient to induce the desired response in a mammal. This amount can be determined by routine clinical and experimental trials known in the art.

By mammal, we mean any mammal but especially a human.

As is clear from the examples of activators and NF-κB inducers indicated herein, it is preferred that the activator or inducer enters the cell and acts within the cell, ie acts as an intracellular activator or NF-κB inducer, for example an intracellular modulator of intracellular signalling events leading to APC or NF-κB activation.

It will be appreciated that inhibitors of inhibitors of NF-κB may act as inducers of NF-κB. Thus, for example, antibodies or antisense molecules or ribozymes that block IκBα function or expression may act as inducers of NF-κB.

Ribozymes which may be encoded in the genomes of the viruses or virus-like particles herein disclosed are described in Cech and Herschlag “Site-specific cleavage of single stranded DNA” U.S. Pat. No. 5,180,818; Altman et al “Cleavage of targeted RNA by RNAse P” U.S. Pat. No. 5,168,053, Cantin et al “Ribozyme cleavage of HIV-1 RNA” U.S. Pat. No. 5,149,796; Cech et al “RNA ribozyme restriction endoribonucleases and methods”, U.S. Pat. No. 5,116,742; Been et al “RNA ribozyme polymerases, dephosphorylases, restriction endonucleases and methods”, U.S. Pat. No. 5,093,246; and Been et al “RNA ribozyme polymerases, dephosphorylases, restriction endoribonucleases and methods; cleaves single-stranded RNA at specific site by transesterification”, U.S. Pat. No. 4,987,071, all incorporated herein by reference.

Preferably the activator of APC function, or inducer of NFκB, or MyD88 molecule, is encoded by a nucleic acid sequence, for example within a vector, such as an adenovirus. The nucleic acid sequence encoding the activator, inducer or molecule is preferably operatively linked to regulatory elements necessary for expression of said sequence. Such vectors may be used for gene therapy to enable the nucleic acid sequence encoding the activator, inducer or molecule to be inserted into the body of a mammal. Methods of gene therapy, such as by using an adenovirus, are known in the art. The vector may also comprise a nucleic acid sequence encoding an allergen.

It may be desirable to supply both an activator, inducer or MyD88 molecule and an allergen to the desired cell. It is preferred that either the activator, inducer or MyD88 molecule or allergen, preferably both, are supplied to the desired cell by means of expression in the desired cell.

The allergen may be a fragment of a naturally occurring allergen, for example a fragment that is useful in modulating the T cell response whilst avoiding augmenting the allergic B cell response. Such fragments are discussed in papers by J R Lamb, R O'Hehir or M Gefter, for example in Wallner B P & Gefter M L (1996) Peptide therapy for treatment of allergic diseases. Clin Immunol Immunopathol 1996 August; 80(2):105-109 and Lamb & O'Hehir (1996) Peptide mediated regulation of allergen specific immune response Adv Exp Med Biol 409, 451-456. Suitable fragments may also be described in WO99/34826.

The term “allergen” will be well known to those skilled in the art. For example, it encompasses a substance which provokes an immune response in a mammal resulting in production of antibodies of the IgE class, and/or which triggers an allergic reaction in a mammal. An allergic response may involve release of mediators such as histamine, leukotrienes, platelet activating factors, chemotactic and enzymes from mast cells, as well known to those skilled in the art. Allergens may be or comprise a polypeptide, lipid, carbohydrate or combinations thereof. Typically allergens may be polypeptides.

“Operatively linked” refers to juxtaposition such that the normal function of the components can be performed. Thus, a coding sequence “operatively linked” to regulatory elements refers to a configuration wherein the nucleic acid sequence encoding the activator, molecule or inducer of NF-κB can be expressed under the control of the regulatory sequences.

“Regulatory sequences” refers to nucleic acid sequences necessary for the expression of an operatively linked coding sequence in a particular host organism. For example, the regulatory sequences which are suitable for eukaryotic cells are promotors, polyadenylation signals, and enhancers.

“Vectors” means a DNA molecule comprising a single strand, double strand, circular or supercoiled DNA. Suitable vectors include retroviruses, adenoviruses, adeno-associated viruses, pox viruses and bacterial plasmids. Retroviral vectors are retroviruses that replicate by randomly integrating their genome into that of the host. Suitable retroviral vectors are described in WO 92/07573.

Adenovirus is a linear double-standard DNA Virus. Suitable adenoviral vectors are described in Rosenfeld et al, Science, 1991, Vol. 252, page 432.

Adeno-associated viruses (AAV) belong to the parvo virus family and consist of a single strand DNA or about 4-6 KB.

Pox viral vectors are large viruses and have several sites in which genes can be inserted. They are thermostable and can be stored at room temperature. Safety studies indicate that pox viral vectors are replication-defective and cannot be transmitted from host to host or to the environment.

Targeting the vaccine to specific cell populations, for example antigen presenting cells, may be achieved, for example, either by the site of injection, use of targeting vectors and delivery systems, or selective purification of such a cell population from the patient and ex vivo administration of the peptide or nucleic acid (for example dendritic cells may be sorted as described in Zhou et al (1995) Blood 86, 3295-3301; Roth et al (1996) Scand. J. Immunology 43, 646-651). In addition, targeting vectors may comprise a tissue- or tumour-selective promoter which directs expression of the antigen at a suitable place.

Although the genetic construct can be DNA or RNA it is preferred if it is DNA.

Preferably, the genetic construct is adapted for delivery to a human cell.

Means and methods of introducing a genetic construct into a cell in or removed from an animal body are known in the art. For example, the constructs of the invention may be introduced into the cells by any convenient method, for example methods involving retroviruses, so that the construct is inserted into the genome of the (dividing) cell. Targeted retroviruses are available for use in the invention; for example, sequences conferring specific binding affinities may be engineered into pre-existing viral env genes (see Miller & Vile (1995) Faseb J. 9, 190-199 for a review of this and other targeted vectors for gene therapy).

Preferred retroviral vectors are lentiviral vectors such as those described in Verma & Somia (1997) Nature 389, 239-242.

It will be appreciated that retroviral methods, such as those described below, may only be suitable when the cell is a dividing cell. For example, in Kuriyama et al (1991) Cell Struc. and Func. 16, 503-510 purified retroviruses are administered. Retroviral DNA constructs which encode the desired polypeptide(s) may be made using methods well known in the art. To produce active retrovirus from such a construct it is usual to use an ecotropic psi2 packaging cell line grown in Dulbecco's modified Eagle's medium (DMEM) containing 10% foetal calf serum (FCS). Transfection of the cell line is conveniently by calcium phosphate co-precipitation, and stable transformants are selected by addition of G418 to a final concentration of 1 mg/ml (assuming the retroviral construct contains a neo^{R} gene). Independent colonies are isolated and expanded and the culture supernatant removed, filtered through a 0.45 μm pore-size filter and stored at −70°. For the introduction of the retrovirus into the target cells, it is convenient to inject directly retroviral supernatant to which 10 μg/ml Polybrene has been added. The injection may be made into the area in which the target cells are present, for example subcutaneously.

Other methods involve simple delivery of the construct into the cell for expression therein either for a limited time or, following integration into the genome, for a longer time. An example of the latter approach includes liposomes (Nässander et al (1992) Cancer Res. 52, 646-653). Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is polylysine.

Bacterial delivery is described in Dietrich (2000) Antisense Nucleic Acid Drug Delivery 10, 391-399.

The DNA may also be delivered by adenovirus wherein it is present within the adenovirus particle, for example, as described below.

In the second of these methods, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the target cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

“Naked DNA” and DNA complexed with cationic and neutral lipids may also be useful in introducing the DNA of the invention into cells of the patient to be treated. Non-viral approaches to gene therapy are described in Ledley (1995) Human Gene Therapy 6, 1129-1144. Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct of the invention to a cell. Thus, it will be appreciated that a further aspect of the invention provides a virus or virus-like particle comprising a genetic construct of the invention. Other suitable viruses or virus-like particles include HSV, AAV, vaccinia, lentivirus and parvovirus.

Preferred vectors include lentivirus vectors and adenoviral vectors, for example vectors similar to those described in Foxwell et al (2000) Ann Rheum Dis 59 Suppl 1, I54-59 or Bondeson et al (2000) J Rheumatol 27(9), 2078-2089.

Vectors comprising nucleic acid encoding an activator, molecule or NF-κB inducer may be introduced into a mammal in the form of liposomes in a manner known in the art. Alternatively, liposomes may be used in the form of aerosols in order to access the body by means of the mucus membrane or lung. Such techniques are known in the art.

Immunoliposomes (antibody-directed liposomes) are especially useful in targeting to cell types which over-express a cell surface protein for which antibodies are available, as is possible with dendritic cells or precursors, for example using antibodies to CD1, CD14 or CD83 (or other dendritic cell or precursor cell surface molecule, as indicated above). For the preparation of immuno-liposomes MPB-PE (N-[4-(p-maleimidophenyl)butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the DNA or other genetic construct of the invention for delivery to the target cells, for example, by forming tie said liposomes in a solution of the DNA or other genetic construct, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from unconjugated antibodies by ultracentrifugation at 80 000×g for 45 min. Immunoliposomes may be injected, for example intraperitoneally or directly into a site where the target cells are present, for example subcutaneously. Naked DNA encoding an activator of APC function, MyD88 molecule or inducer of NF-κB, in the form of a DNA vaccine, may also be used in modulating the T_{H1}:T_{H2} ratio of an immune response or for treating a patient with or at risk of allergy.

As noted above, an alternative activator or inducer of NFκB is the use of anti-sense nucleic acid to an IκB sequence. Such an anti-sense nucleic acid comprises a nucleic acid sequence which is capable of binding to an IκB nucleic acid sequence, inhibiting transcription of the IκB sequence. Methods of producing anti-sense nucleic acid per se are known in the art.

Antisense oligonucleotides are single-stranded nucleic acids, which can specifically bind to a complementary nucleic acid sequence. By binding to the appropriate target sequence, an RNA-RNA, a DNA-DNA, or RNA-DNA duplex is formed. These nucleic acids are often termed “antisense” because they are complementary to the sense or coding strand of the gene. Further, formation of a triple helix has proven possible where the oligonucleotide is bound to a DNA duplex (triple helix-forming oligonucleotide; TFO). It was found that oligonucleotides could recognise sequences in the major groove of the DNA double helix. A triple helix was formed thereby. This suggests that it is possible to synthesise a sequence-specific molecules which specifically bind double-stranded DNA via recognition of major groove hydrogen binding sites.

By binding to the target nucleic acid, the above oligonucleotides can inhibit the function of the target nucleic acid. This could, for example, be a result of blocking the transcription, processing, poly(A)addition, replication, translation, or promoting inhibitory mechanisms of the cells, such as promoting RNA degradations.

Antisense oligonucleotides are prepared in the laboratory and then introduced into cells, for example by microinjection or uptake from the cell culture medium into the cells, or they are expressed in cells after transfection with plasmids or retroviruses or other vectors carrying an antisense gene. Antisense oligonucleotides were first discovered to inhibit viral replication or expression in cell culture for Rous sarcoma virus, vesicular stomatitis virus, herpes simplex virus type 1, simian virus and influenza virus. Since then, inhibition of mRNA translation by antisense oligonucleotides has been studied extensively in cell-free systems including rabbit reticulocyte lysates and wheat germ extracts. Inhibition of viral function by antisense oligonucleotides has been demonstrated in vitro using oligonucleotides which were complementary to the AIDS HIV retrovirus RNA (Goodchild, J. 1988 “Inhibition of Human Immunodeficiency Virus Replication by Antisense Oligodeoxynucleotides”, Proc. Natl. Acad. Sci. (USA) 85(15), 5507-11). The Goodchild study showed that oligonucleotides that were most effective were complementary to the poly(A) signal; also effective were those targeted at the 5′ end of the RNA, particularly the cap and 5′ untranslated region, next to the primer binding site and at the primer binding site. The cap, 5′ untranslated region, and poly(A) signal lie within the sequence repeated at the ends of retrovirus RNA (R region) and the oligonucleotides complementary to these may bind twice to the RNA.

Typically, antisense oligonucleotides are 15 to 35 bases in length. For example, 20-mer oligonucleotides have been shown to inhibit the expression of the epidermal growth factor receptor mRNA (Witters et al, Breast Cancer Res Treat 53:41-50 (1999)) and 25-mer oligonucleotides have been shown to decrease the expression of adrenocorticotropic hormone by greater than 90% (Frankel et al, J Neurosurg 91:261-7 (1999)). However, it is appreciated that it may be desirable to use oligonucleotides with lengths outside this range, for example 10, 11, 12, 13, or 14 bases, or 36, 37, 38, 39 or 40 bases.

The anti-sense nucleic acid may be encoded by a suitable vector, for example of the type discussed above.

The activator or inducer may be an antibody, by which term is included antibody fragments or antibody-like molecules, as well known to those skilled in the art. For example, the antibody may bind to MyD88 or to a binding partner of MyD88. For example, the antibody may bind to the DD of MyD88 (and/or to the DD of a binding partner of MyD88), and may disrupt binding of MyD88 to a DD of a binding partner of MyD88. Alternatively, the antibody may bind to the Toll domain of MyD88 (and/or to the Toll domain of a binding partner of MyD88), and may disrupt binding of MyD88 to a Toll domain of a binding partner of MyD88. The antibody may preferably bind to an epitope of MyD88 that comprises the residue equivalent to Phe56 of wild-type mouse MyD88.

The activator or inducer may alternatively be, for example, an anti-IκB vaccine or an antibody against IκB or fragment thereof such as an Fv. The vaccine or antibody may be against any suitable part of IκB (or other inhibitor of NFκB) providing it results in the induction or activation of NF-κB.

By an antibody is included an antibody or other immunoglobulin, or a fragment or derivative thereof, as discussed further below.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties. Effector functions of whole antibodies, such as complement binding, are removed Fab, Fv, ScFv and dAb antibody fragments can all be expressed in and secreted from E. coli, thus allowing the facile production of large amounts of the said fragments. Fragments may also be expressed in cells of the patient.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

Preferably, the antibody has an affinity for the epitope of between about 10⁵.M⁻¹ to about 10¹².M⁻¹, more preferably at least 10⁸.M¹.

Antibodies reactive towards a chosen polypeptide may be made by methods well known in the art. In particular, the antibodies may be polyclonal or monoclonal.

Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799). Suitably prepared non-human antibodies cam be “humanized” in known ways, for example by inserting the CDR regions of mouse antibodies into the framework of human antibodies.

Techniques for preparing antibodies are well known to those skilled in the art, for example as described in Harlow, E D & Lane, D “Antibodies: a laboratory manual” (1988) New York Cold Spring Harbor Laboratory. Suitable antibodies and techniques for preparing suitable antibodies to MyD88 may be described in (5).

The antibody (particularly antibody fragment) may be joined to a moiety that facilitates uptake of the antibody by a cell, for example a DC. For example, the antibody may be linked to a lipophilic molecule or polypeptide domain that is capable of promoting cellular uptake of the molecule or the interacting polypeptide, as known to those skilled in the art. Thus, the moiety may derivable from the Antennapedia helix 3 (Derossi et al (1998) Trends Cell Biol 8, 84-87), or from sequences of HIV, generally tat, that permit entry into cells. Alternatively, a polynucleotide, for example cDNA, encoding the antibody may be delivered in a vector, permitting expression of the antibody the cell, as indicated above.

Preferred NF-κB inducers include NFκB or Rel B or other NF-κB subunit, a TRAF (including TRAF 2, 3, 4, 5, 6, for example TRAF2, TRAF5 or TRAF6), TRADD, NIK, IKK1, IKK2, IKKε TAK1, PKR, NAK, MEKK, p65/relA, c-rel, rel B, p38MAK, p54JNK, p42/44Erk, a MEK (including MEK 1, 2, 3, 4, 5, 6, 7,) or a MEKK (including MEKK1, 2, 3). Fragments and muteins of such inducers capable of inducing an NF-κB may also be used. The inducers may be encoded by suitable vectors, as described above, and introduced into the cells of a patient to be treated.

As noted above, a dominant negative mutant of MyD88 (Myd88dn, ie capable of inhibiting signalling by wild-type MyD88 molecules, for example in a cell in which wild-type and inhibitory MyD88 molecules are present) may be useful in modulating the T_{H1}:T_{H2} ratio of an immune response, and may act as an NF-κB inducer. The inhibition of signalling may arise from blocking interaction of endogenous wild-type MyD88 with a binding partner of the endogenous MyD88, for example a Toll-Like Receptor (TLR). The dominant negative mutant may be MyD881pr (Burns et al (1998) J Biol Chem 273(20), 12203-12209) or a fragment of MyD88 lacking a death domain (see Burns et al (1998) and references reviewed therein). The MyD88 (myeloid differentiation protein) is considered to have a modular organisation consisting of an N-terminal death domain (DD) separated by a short linker from a C-terminal Toll domain (reviewed in Burns et al (1998)). The N-terminal DD is related to a motif of approximately 90 amino acids that is considered to mediate protein-protein interactions with other DD sequences forming either homo- or heterodimers (Boldin et al (1995) J Biol Chem 270, 387-391).

The inhibitory MyD88 molecule may be a MyD88 molecule that is less able than MyD88, preferably substantially unable, to bind to a DD, for example the DD of MyD88 or of IRAK. For example, the inhibitory MyD88 may be less able than MyD88, preferably substantially unable, to dimerise via the DD. The inhibitory MyD88 molecule may be a truncated version of MyD88, for example a MyD88 molecule in which all or part of the domain termed the Death Domain is deleted. It may be a mutated MyD88 molecule, for example a MyD88 molecule that is mutated in the DD, for example with a non-conservative mutation. For example, it may be mutated at the position equivalent to Phe56 of full length mouse MyD88, for example to Asn. It may be the mutated MyD88 molecule termed MyD881pr, as noted above in which the N terminal 53 amino acids of MyD88 are also absent Burns et al (1998) J. Biol. Chem. 273, 12203-12209. MyD881pr has a point mutation (F56N; mouse sequence numbering) when compared with wild-type MyD88, for example mouse wild-type MyD88. This point mutation is in the DD and prevents dimerisation of the DD (Burns et al (1998)). The mutation corresponds to the 1pr^{cp} mutation known to abolish cytotoxic signalling of Fas, probably by disrupting the conformation of the DD domain (Nagata (1994) Semin Immunol 6, 3-8; Huang et al (1996) Nature 384, 638-641).

The constructs for the wild-type MyD88 and dominant negative MyD88 (MyD88-1pr) has been published (Burns K. et al J. Biol Chem 1998) but MyD88-1pr is wrongly described as a single amino acid mutation in its death domain, where Phe⁵⁶ is mutated to Asn. This mutation corresponds to the 1pr^{cp} mutation present in the death domain of Fas ligand which abolishes its downstream signalling by disrupting the conformation of the death domain. Actually, in addition to the point mutation there is a deletion in its N-terminal domain of 53 amino acids (1-159 base pars of the genebank sequence are missing). This deletion results in part of the death domain missing.

It is preferred that the inhibitory MyD88 comprises a functional Toll domain, ie a Toll domain that is capable of interacting with a Toll domain, for example the Toll domain of a wild-type MyD88, for example wild-type human or mouse MyD88 or a TLR. It is preferred that the inhibitory MyD88 comprises the full-length MyD88 Toll domain. A full-length Toll domain may be necessary for Toll-Toll domain interaction.

Methods of measuring protein-protein interactions (and their enhancement or disruption) will be well known to those skilled in the art. Suitable methods of measuring DD and Toll-Toll interactions are also described in Burns et at (1998). Suitable methods may include, for example, yeast two-hybrid interactions, co-purification, ELISA, co-immunoprecipitation, fluorescence resonance energy transfer (FRET) techniques and surface plasmon resonance methods. Thus, a MyD88 molecule may be considered capable of binding to or interacting with a DD or Toll domain if an interaction may be detected between the said MyD88 polypeptide and a polypeptide comprising a DD or Toll domain by ELISA co-immunoprecipitation or surface plasmon resonance methods or by a yeast two-hybrid interaction or copurification method. The preferred method is surface plasmon resonance.

A wild-type MyD88 molecule (which term includes a molecule which retains properties of naturally occurring MyD88) may also be useful in modulating the T_{H1}:T_{H2} ratio of an immune response and in treating a patient with or at risk of allergy. The wild-type MyD88 molecule may be a MyD88 molecule that retains the ability of naturally occurring MyD88 to bind to a DD, for example the DD of MyD88 or of IRAK. It may retain the ability of naturally occurring MyD88 to activate one or more MAPK kinase pathways, for example the p38, p54/JNK and/or p42/44Erk pathway. It preferably has a functional Toll domain and a functional DD (ie has a domain that is capable of binding to a DD). It is preferred that the MyD88 Toll domain and/or DD are unmutated, ie that any mutation lies outside these domains.

It is preferred that the MyD88 has the sequence indicated in Hardiman et al (1996) Oncogene 13, 2467-2475; or Bonnert et al (1997) FEBS Lett. 402, 81-84; or Hardiman et al (1997) Genomics 45, 332-339, all of which are human. The human sequence is also given in Gen Bank Accession No. NM-002468.

Human MyD88 is 82% identical in amino acid sequence to the mouse MyD88.

It is preferred that any mutation is a conservative substitution, as well known to those skilled in the art. By “conservative substitutions” is intended combinations such as Gly, Ala; Val, Ile, Leu; Asp, Glu; Asn, Gln; Ser, Thr; Lys, Arg; and Phe, Tyr. Mutations may be made using the methods of protein engineering and site-directed mutagenesis as well known to those skilled in the art.

The three-letter and one-letter amino acid code of the IUPAC-IUB Biochemical Nomenclature Commission is used herein. The sequence of polypeptides are given N-terminal to C-terminal as is conventional. It is preferred that the amino acids are L-amino acids, but they may be D-amino acid residues.

Preliminary work indicates that MEKK1 can induce NF-κB and enhance APC such as DC function. It is preferred that the inducer is capable of inducing NF-κB in DC or precursors thereof.

Thus, inducers or enhancers of APC function may be useful in an anti-allergy vaccine production.

It is preferred that the patient or cell is, has or will be supplied with an allergen (by which is included a fragment of a naturally occurring allergen, as will be well known to those skilled in the art). However, this is not considered to be essential. Administration of the activator, inducer or MyD88 molecule and environmental exposure to the allergen may be sufficient.

Nevertheless, it is preferred that the patient (or APCs from the patient) is supplied with both the activator, inducer or MyD88 molecule, and an allergen. The supply of both agents may be achieved by administering a single (chimaeric) molecule, or a composition comprising both agents, or by administering more than one composition, either simultaneously or temporally separated. It is preferred that the activator, inducer or MyD88 molecule is supplied before or simultaneously (ie within about 1 hour, preferably 30, 20, 10 or 5 minutes) with the allergen.

It will be appreciated that more than one administration of each agent may advantageously be supplied to the patient. For example a “booster” administration of allergen and/or activator, inducer or MyD88 molecule may be desirable or necessary for optimal efficacy, as known to those skilled in the art and discussed further in the Examples.

A further aspect of the invention provides a molecule comprising (1) a portion (modulating portion) comprising or encoding an activator or inducer or MyD88 molecule as defined above, for example an intracellular intracellular activator of antigen-presenting cell (APC), such as DC, function and (2) a portion comprising or encoding an allergen. In a preferred embodiment, the invention provides a recombinant polynucleotide comprising (1) a portion (modulating portion) encoding an activator or inducer or MyD88 molecule as defined above and (2) a portion encoding an allergen.

A further aspect of the invention provides a kit of parts, composition or a chimaeric molecule comprising (1) a portion (modulating portion) comprising or encoding an activator or inducer or Myd88 molecule as defined above and (2) a portion comprising or encoding an allergen.

Preferably, the molecule is or comprises a DNA vaccine encoding an allergen and an enhancer of APC, such as DC, function, inducer of NFκB or MyD88 molecule, as discussed above. The modulator, for example enhancer of APC, such as DC, function may be an intracellular signalling molecule or derivative thereof which retains or has enhanced intracellular signalling activity. It is preferred if the derivative is one which retains or enhances DC function. It is preferably an activator/inducer of NF-κB. It may be NF-κB or a component thereof. The DNA vaccine may comprise a recombinant polynucleotide comprising a portion encoding the activator of APC, such as DC function, inducer of NFκB or MyD88 molecule and a portion encoding an allergen. The activator, inducer or molecule and allergen may be transcribed from a single promoter with an internal ribosome entry site (IRES) for the second coding sequence. Alternatively, the signalling molecule and allergen may be transcribed from separate promoters. Alternatively, the allergen may be encoded on a separate polynucleotide molecule; this is less preferred.

Preferred enhancers are NFκB and a dominant negative mutant of MyD88, for example MyD881pr.

It will be appreciated that the preferred enhancers/inducers as described above may be used in the vaccines of the invention.

The allergen portion may comprise more than one copy of one or more epitopes. For example, it may comprise a single copy of a single epitope-forming amino acid sequence, for example a sequence of between about 8 and 30 amino acids, preferably about 10 to 18 amino acids, still more preferably about 15 amino acids in length. It may comprise multiple copies of such an epitope-forming sequence, or single or multiple copies of at least two different epitope-forming sequences. The antigenic sequences may be concatenated to form a domain-like structure, or may be disposed at different points in a carrier polypeptide. The polynucleotide may encode one or several different allergen molecules, each of which may have one or more antigenic portions or epitopes.

As discussed further below, the allergen may be an allergen associated with asthma, rhinitis, atopic dermatitis or hayfever.

The invention also includes DNA vaccines encoding an activator, inducer of NF-κB or MyD88 molecule (as defined above) and an allergen for use in the invention. Such vaccines could include DNA sequences incorporating an allergen of interest. In addition, such vaccines would also include an activator of APCs or NFκB, or MyD88 molecule, possibly two or more activators and/or MyD88 molecules, for maximum effect. Both allergen and activator would be under the control of suitable promoter sequences to regulate expression of allergen and activators. An alternative method of modulating the immune response may be to provide a vector comprising a nucleic acid sequence encoding an APC activator or NF-κB inducer or MyD88 molecule operatively linked to regulatory elements necessary for expressing said sequence. The vector may comprise an inducible promoter to enable an increased immune response to be produced by the increased activation of APCs or NF-κB.

The use of recombinant polyepitope vaccines for the delivery of multiple CD8 CTL epitopes is described in Thomson et al (1996) J. Immunol. 157, 822-826 and WO 96/03144, both of which are incorporated herein by reference. In relation to the present invention, it may be desirable to include in a single vaccine, a peptide (or a nucleic acid encoding a peptide) wherein the peptide includes, in any order, one or more antigenic amino acid sequences (for example each of between about 8 and 18 amino acids in length) derived from an allergen, and a CD4 T cell-stimulating epitope (such as from tetanus toxoid). Such “bead-on-a-string” vaccines are typically DNA vaccines.

The allergen may comprise an epitope present in a naturally occurring allergen, for example in pollen, house dust or animal dander, as discussed further below.

The epitope may be a T-cell epitope ie an epitope that is capable of inducing a T-cell response (TH-1 response), preferably a CD8+ cytotoxic T-cell response, as well known to those skilled in the art.

According to current immunological theories, a carrier function should be present in any immunogenic formulation in order to stimulate, or enhance stimulation of, the immune system. The epitope(s) as defined above in relation to the preceding aspects of the invention may be associated, for example by cross-linking, with a separate carrier, such as serum albumins, myoglobins, bacterial toxoids and keyhole limpet haemocyanin. More recently developed carriers which induce T-cell help in the immune response include the hepatitis-B core antigen (also called the nucleocapsid protein), presumed T-cell epitopes such as Thr-Ala-Ser-Gly-Val-Ala-Glu-Thr-Thr-Asn-Cys, beta-galactosidase and the 163-171 peptide of interleukin-1. The latter compound may variously be regarded as a carrier or as an adjuvant or as both.

Alternatively, several copies of the same or different epitope may be cross-linked to one another; in this situation there is no separate carrier as such, but a carrier function may be provided by such cross-linking. Suitable cross-linking agents include those listed as such in the Sigma and Pierce catalogues, for example glutaraldehyde, carbodiimide and succinimidyl 4-(N-maleimidomethyl)cyclohexane-1-carboxylate, the latter agent exploiting the —SH group on the C-terminal cysteine residue (if present). Any of the conventional ways of cross-linking polypeptides may be used, such as those generally described in O'Sullivan et al Anal. Biochem. (1979) 100, 100-108. For example, the first portion may be enriched with thiol groups and the second portion reacted with a bifunctional agent capable of reacting with those thiol groups, for example the N-hydroxysuccinimide ester of iodoacetic acid (NHIA) or N-succinimidyl-3-(2-pyridyldithio)propionate (SPDP), a heterobifunctional cross-linking agent which incorporates a disulphide bridge between the conjugated species. Amide and thioether bonds, for example achieved with m-maleimidobenzoyl-N-hydroxysuccinimide ester, are generally more stable in vivo than disulphide bonds.

Further useful cross-linking agents include S-acetylthioglycolic acid N-hydroxysuccinimide ester (SATA) which is a thiolating reagent for primary amines which allows deprotection of the sulphydryl group under mild conditions (Julian et al (1983) Anal. Biochem. 132, 68), dimethylsuberimidate dihydrochloride and N,N′-o-phenylenedimalemide.

If the polypeptide is prepared by expression of a suitable nucleotide sequence in a suitable host, then it may be advantageous to express the polypeptide as a fusion product with a peptide sequence which acts as a carrier. Kabigen's “Ecosec” system is an example of such an arrangement.

Epitopes from different biological sources (for example different allergen molecules, either from the same or different organisms) may be linked to other allergens to provide a dual effect.

By epitopes is included mimotopes, as well known to those skilled in the art.

The activator, inducer, MyD88 molecule or allergen may be a peptidomimetic compound, for example a peptidomimetic compound corresponding to a polypeptide inhibitor or inducer discussed above.

The term “peptidomimetic” refers to a compound that mimics the conformation and desirable features of a particular peptide as a therapeutic agent, but that avoids potentially undesirable features. For example, morphine is a compound which can be orally administered, and which is a peptidomimetic of the peptide endorphin.

Therapeutic applications involving peptides may be limited, due to lack of oral bioavailability and to proteolytic degradation. Typically, for example, peptides are rapidly degraded in vivo by exo- and endopeptidases, resulting in generally very short biological half-lives. Another deficiency of peptides as potential therapeutic agents is their lack of bioavailability via oral administration. Degradation of the peptides by proteolytic enzymes in the gastrointestinal tract is likely to be an important contributing factor. The problem is, however, more complicated because it has been recognised that even small, cyclic peptides which are not subject to rapid metabolite inactivation nevertheless exhibit poor oral bioavailability. This is likely to be due to poor transport across the intestinal membrane and rapid clearance from the blood by hepatic extraction and subsequent excretion into the intestine. These observations suggest that multiple amide bonds may interfere with oral bioavailability. It is thought that the peptide bonds linking the amino acid residues in the peptide chain may break apart when the peptide drug is orally administered.

There are a number of different approaches to the design and synthesis of peptidomimetics. In one approach, such as disclosed by Sherman and Spatola, J. Am. Chem. Soc., 112: 433 (1990), one or more amide bonds have been replaced in an essentially isoteric manner by a variety of chemical functional groups. This stepwise approach has met with some success in that active analogues have been obtained. In some instances, these analogues have been shown to possess longer biological half-lives than their naturally-occurring counterparts. Nevertheless, this approach has limitations. Successful replacement of more than one amide bond has been rare. Consequently, the resulting analogues have remained susceptible to enzymatic inactivation elsewhere in the molecule. When replacing the peptide bond it is preferred that the new linker moiety has substantially the same charge distribution and substantially the same planarity as a peptide bond.

Retro-inverso peptidomimetics, in which the peptide bonds are reversed, can be synthesised by methods known in the art, for example such as those described in Mézière et al (1997) J. Immunol. 159 3230-3237. This approach involves making pseudopeptides containing changes involving the backbone, and not the orientation of side chains. Retro-inverse peptides, which contain NH—CO bonds instead of CO—NH peptide bonds, are much more resistant to proteolysis.

In another approach, a variety of uncoded or modified amino acids such as D-amino acids and N-methyl amino acids have been used to modify mammalian peptides. Alternatively, a presumed bioactive conformation has been stabilised by a covalent modification, such as cyclisation or by incorporation of γ-lactam or other types of bridges. See, eg. Veber et al, Proc. Natl. Acad. Sci. USA, 75:2636 (1978) and Thursell et al, Biochem. Biophys. Res. Comm., 111:166 (1983).

A common theme among many of the synthetic strategies has been the introduction of some cyclic moiety into a peptide-based framework. The cyclic moiety restricts the conformational space of the peptide structure and this frequently results in an increased affinity of the peptide for a particular biological receptor. An added advantage of this strategy is that the introduction of a cyclic moiety into a peptide may also result in the peptide having a diminished sensitivity to cellular peptidases.

One approach to the synthesis of cyclic stabilised peptidomimetics is ring closing metathesis (RCM). This method involves steps of synthesising a peptide precursor and contacting it with a RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Polypeptides in which one or more of the amino acid residues are chemically modified, before or after the polypeptide is synthesised, may be used as antigen providing that the function of the polypeptide, namely the production of a specific immune response in vivo, remains substantially unchanged. Such modifications include forming salts with acids or bases, especially physiologically acceptable organic or inorganic acids and bases, forming an ester or amide of a terminal carboxyl group, and attaching amino acid protecting groups such as N-t-butoxycarbonyl. Such modifications may protect the polypeptide from in vivo metabolism.

Either or both portions in these aspects of the invention may further comprise a translocating portion and/or a cell binding portion. The cell binding portion is preferably capable of binding to a dendritic cell or precursor thereof. The translocating portion may aid in internalisation of the molecule or at least the allergen portion and preferably the signalling enhancing portion. Thus, exogenously applied peptides may be linked to a HIV tat peptide. This may direct them into the MHC Class I pathway for presentation by CTL (see, for example, Kim et al (1997) J. Immunol. 159, 1666-1668. Chimaeric molecules which may be adapted in accordance with the present invention are described in WO95/31483.

Dendritic cells may be characterised by expression of the CD80, CD86, CD40, CD1a, HLA-DR and/or CD83 cell surface molecules. Immature dendritic cells may be CD34⁺ or CD14⁺. Thus, the cell biding portion may be capable of binding to one or more of these cell surface molecules (for example, an antibody capable of binding to such a molecule).

Immature DCs show increased antigen capture and processing. They show high intracellular MHC Class I and II; increased endocytosis and phagocytosis; high CCR1, CCR5 and CCR6; low CCR7; high CD68; low CD40, CD54, CD80, CD83, and CD86; and no DC-LAMP.

Mature DCs show increased antigen processing. They show high surface MHC Class I and II; low endocytosis and phagocytosis; low CCR1, CCR5 and CCR6; high CCR7; low CD68; high CD40, CD54, CD58, CD80, CD83 and CD86; high DC-LAMP; and high p55 fascin.

Such a cell binding portion may be useful in directing any inhibitor or activator as herein described, for example nucleic acid, DNA vaccine or antibody, to an APC such as a DC or immature DC.

Preferably, the polynucleotide or DNA vaccine is capable of expressing the encoded antisense molecule or polypeptide(s) in the patient, still more preferably in an APC such as a DC or immature DC of the patient. The antisense molecule or polypeptide(s), for example NF-κB inducer/activator, or allergen, as appropriate, may be expressed from any suitable polynucleotide (genetic construct) as is described herein and delivered to the patient. Typically, the genetic construct which expresses the antisense molecule or polypeptide comprises the said polypeptide coding sequence operatively linked to a promoter which can express the transcribed polynucleotide (eg mRNA) molecule in a cell of the patient, which may be translated to synthesise the said polypeptide. Suitable promoters will be known to those skilled in the art, and may include promoters for ubiquitously expressed, for example housekeeping genes or for tissue-specific genes, depending upon where it is desired to express the said polypeptide (for example, in dendritic cells or precursors thereof). Preferably, a dendritic cell or dendritic precursor cell-selective promoter is used, but this is not essential, particularly if delivery or uptake of the polynucleotide is targeted to the selected cells ie dendritic cells or precursors.

Promoters that may be selective for dendritic cells may be promoters from the CD36 or CD83 genes.

Targeting the vaccine to specific cell populations, for example antigen presenting cells, may be achieved, for example, either by the site of injection, use of targeting vectors and delivery systems, or selective purification of such a cell population from the patient and ex vivo administration of the peptide or nucleic acid (for example dendritic cells may be sorted as described in Zhou et al (1995) Blood 86, 3295-3301; Roth et al (1996) Scand. J. Immunology 43, 646-651). In addition, targeting vectors may comprise a tissue- or tumour-specific promoter which directs expression of the allergen at a suitable place.

As noted above, it may be desirable to use an inducible promoter. It will be appreciated that it may be desirable to be able to regulate temporally expression of the polypeptide(s) (for example NF-κB activator/inducer) in the cell. Thus, it may be desirable that expression of the polypeptide(s) is directly or indirectly (see below) under the control of a promoter that may be regulated, for example by the concentration of a small molecule that may be administered to the patient when it is desired to activate or repress (depending upon whether the small molecule effects activation or repression of the said promoter) expression of the polypeptide. It will be appreciated that this may be of particular benefit if the expression construct is stable ie capable of expressing the polypeptide (in the presence of any necessary regulatory molecules) in the said cell for a period of at least one week, one, two, three, four, five, six, eight months or more. A preferred construct of the invention may comprise a regulatable promoter. Examples of regulatable promoters include those referred to in the following papers: Rivera et at (1999) Proc Natl Acad Sci USA 96(15), 8657-62 (control by rapamycin, an orally bioavailable drug, using two separate adenovirus or adeno-associated virus (AAV) vectors, one encoding an inducible human growth hormone (hGH) target gene, and the other a bipartite rapamycin-regulated transcription factor); Magari et al (1997) J Clin Invest 100(11), 2865-72 (control by rapamycin); Bueler (1999) Biol Chem 380(6), 613-22 (review of adeno-associated viral vectors); Bohl et al (1998) Blood 92(5), 1512-7 (control by doxycycline in adeno-associated vector); Abruzzese et al (1996) J Mol Med 74(7), 379-92 (reviews induction factors e.g., hormones, growth factors, cytokines, cytostatics, irradiation, heat shock and associated responsive elements). Tetracycline-inducible vectors may also be used. These are activated by a relatively-non toxic antibiotic that has been shown to be useful for regulating expression in mammalian cell cultures. Also, steroid-based inducers may be useful especially since the steroid receptor complex enters the nucleus where the DNA vector must be segregated prior to transcription.

This system may be further improved by regulating the expression at two levels, for example by using a tissue-specific promoter and a promoter controlled by an exogenous inducer/repressor, for example a small molecule inducer, as discussed above and known to those skilled in the art. Thus, one level of regulation may involve linking the appropriate polypeptide-encoding gene to an inducible promoter whilst a further level of regulation entails using a tissue-specific promoter to drive the gene encoding the requisite inducible transcription factor (which controls expression of the polypeptide (for example NF-κB inducer/activator-encoding gene) from the inducible promoter. Control may further be improved by cell-type-specific targeting of the genetic construct.

The methods or constructs of the invention may be evaluated in, for example, dendritic cells generated in vitro, as known to those skilled in the art, before evaluation in whole animals. The methods described in GB9930616.9, filed on 24 Dec. 1999, may also be used in the evaluation of the methods or constructs of the invention.

The genetic constructs of the invention can be prepared using methods well known in the art.

A further aspect of the invention provides vectors, vaccines and antibodies for use in methods of the invention.

A further aspect of the invention provides a pharmaceutical composition comprising a composition or chimaeric molecule or polynucleotide or vaccine of the invention, and a pharmaceutically acceptable carrier.

A further aspect of the invention provides a pharmaceutical composition, polynucleotide, chimaeric molecule or vaccine of the invention for use in medicine.

A further aspect of the invention provides the use of a pharmaceutical composition, polynucleotide, chimaeric molecule or vaccine of the invention in the manufacture of a medicament for treatment of a patient in need of increasing the T_{H1}:T_{H2} ratio of an immune response and/or with or at risk of allergy.

The vaccines and vectors of the invention (therapeutic molecules of the invention) may be formulated with suitable pharmaceutically-acceptable carriers, fillers or other additives. They may be administered by any suitable means such as intra-muscularly, intra-veinally, orally, anally, intra-nasally, etc. Subcutaneous or intramuscular administration may be preferred. The treatment may consist of a single dose or a plurality of doses over a period of time. It will be appreciated that an inducer, for example small molecule inducer as discussed above may preferably be administered orally.

It may be desirable to locally perfuse an area comprising target cells with the suitable delivery vehicle comprising the therapeutic molecule, for example genetic construct, for a period of time; additionally or alternatively the delivery vehicle or therapeutic molecule can be injected directly into accessible areas comprising target cells, for example subcutaneously. Methods of delivering genetic constructs, for example adenovrial vector constructs to cells of a patient will be well known to those skilled in the art.

In particular, an adoptive therapy protocol may be used or a gene gun may be used to deliver the construct to dendritic cells, for example in the skin.

An adoptive therapy approach may include the steps of (1) obtaining antigen presenting cells or precursors thereof, preferably dendritic cells or precursors thereof, from the patient; (2) contacting said antigen presenting cells with an activator, inducer, MyD88 polypeptide (or polynucleotide encoding same), and optionally allergen to which modulation of the immune response is required; or chimaeric molecule or polynucleotide as defined in any one of the preceding claims, ex vivo; and (3) reintroducing the so treated antigen presenting cells into the patient.

Suitably, the dendritic cells are autologous dendritic cells which are pulsed with polypeptide(s), for example a NF-κB activator and an allergen. T-cell therapy using autologous dendritic cells pulsed with peptides from a tumour associated antigen is disclosed in Murphy et al (1996) The Prostate 29, 371-380 and Tjua et al (1997) The Prostate 32, 272-278.

In a further embodiment the antigen presenting cells, such as dendritic cells, are contacted with a polynucleotide which encodes the activator, NF-κB activator/inducer or MyD88 molecule. The polynucleotide may be any suitable polynucleotide and it is preferred that it is capable of transducing the dendritic cell thus resulting in respectively activation of antigen presentation by the antigen presenting cell.

Conveniently, the polynucleotide may be comprised in a viral polynucleotide or virus, as noted above. For example, adenovirus-transduced dendritic cells have been shown to induce antigen-specific antitumour immunity in relation to MUC1 (see Gong et al (1997) Gene Ther. 4, 1023-1028). Similarly, adenovirus-based systems may be used (see, for example, Wan et al (1997) Hum. Gene Ther. 8, 1355-1363); retroviral systems may be used (Specht et al (1997) J. Exp. Med. 186, 1213-1221 and Szabolcs et al (1997) Blood 90, 2160-2167); particle-mediated transfer to dendritic cells may also be used (Tuting et al (1997) Eur. J. Immunol. 27, 2702-2707); and RNA may also be used (Ashley et al (1997) J. Exp. Med. 186, 1177-1182).

The APCs, such as dendritic cells, may be derived from the patient (ie autologous dendritic cells) or (less preferably) from a healthy individual or individuals (MHC matched), treated in vitro as indicated above, followed by adoptive therapy, ie introduction of the so-manipulated dendritic cells in vivo. By “healthy individual” we mean that the individual is generally in good health, preferably has a competent immune system and, more preferably is not suffering from any disease which can be readily tested for, and detected.

Thus, the methods of the invention include methods of adoptive immunotherapy. It is preferred that such methods are not used when the MyD88 molecule is a MyD88wt molecule.

It is preferred if between about 10³ and 10¹¹ DCs are administered to the patient; more preferably between 10⁶ and 10⁷ DCs.

The APCs such as DCs may be administered by any convenient route. It is preferred if the DCs are administered intravenously. It is also preferred if the DCs are administered locally to the site of the disease (such as a tumour or local vial or bacterial infection). Local administration is particularly preferred for cancer. Conveniently, the DCs are administered into an artery that supplies the site of the disease or the tissue where the disease is located.

The cells (or vaccine) may be given to a patient who is being treated for the disease by some other method. Thus, although the method of treatment may be used alone it is desirable to use it as an adjuvant therapy.

The APCs, such as DCs, or vaccine may be administered before, during or after the other therapy.

It is preferred that administrations are not made during a flare-up of the patient's allergy, or when there is any intercurrent disease.

Allergies which may be treatable by the method described herein include allergies to the following allergens: Fel d 1 (the feline skin and salivary gland allergen of the domestic cat Felis domesticus—the amino acid sequence of which is disclosed in WO 91/06571), Der p I, Der p II, Der fI or Der fII (the major protein allergens from the house dust mite dermatophagoides—amino acid sequences disclosed in WO 94/24281).

The invention is applicable substantially to any allergy, including those caused by allergens present in any of the following: grass, tree and weed (including ragweed) pollens; fungi and moulds; foods eg fish, shellfish, crab lobster, peanuts, nuts, wheat gluten eggs and milk; stinging insects eg bee, wasp and hornet and the chirnomidae (non-biting midges); spiders and mites, including the house dust mite; allergens found in the dander, urine, saliva, blood or other bodily fluid of mammals such as cat, dog, cows, pigs, sheep, horse, rabbit, rat, guinea pig, mouse and gerbil; airborne particulates in general; latex; and protein detergent additives.

Allergies to proteins from the following insects may also be treated: housefly, fruit fly, sheep blow fly, screw worm fly, grain weevil, silkworm, honeybee, non-biting midge larvae, bee moth larvae, mealworm, cockroach and larvae of Tenibrio molitor beetle.

The methods of the invention may be used to treat any mammal such as human, dog, cat, horse, cow and the like. Preferably, the methods are used to treat a human patient.

It will be appreciated that the expressed protein is preferaby produced at an appropriate level relative to other proteins involved in APC signalling for optimal functioning.

Whilst it is possible for a therapeutic molecule as described herein, for example a signalling enhancer or inhibitor or construct or molecule, to be administered alone, it is preferable to present it as a pharmaceutical formulation, together with one or more acceptable carriers. The carrier(s) must be “acceptable” in the sense of being compatible with the therapeutic molecule (which may be a nucleic acid or polypeptide) and not deleterious to the recipients thereof. Typically, the carriers will be water or saline which will be sterile and pyrogen free.

Nasal sprays may be useful formulations.

The formulations may conveniently be presented in unit dosage form and may be prepared by any of the methods well known in the art of pharmacy. Such methods include the step of bringing into association the active ingredient (for example, a activator, inducer or MyD88 molecule as defined above, or construct or molecule of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

The construct, for example, can be administered by means of other implants that are commercially available or described in the scientific literature, including liposomes, microcapsules and implantable devices. For example, implants made of biodegradable materials such as polyanhydrides, polyorthoesters, polylactic acid and polyglycolic acid and copolymers thereof, collagen, and protein polymers, or non-biodegradable materials such as ethylenevinyl acetate (EVAc), polyvinyl acetate, ethylene vinyl alcohol, and derivatives thereof can be used to locally deliver the construct. The construct can be incorporated into the material as it is polymerised or solidified, using melt or solvent evaporation techniques, or mechanically mixed with the material. In one embodiment, the construct (including, for example, an antisense oligonucleotide) are mixed into or applied onto coatings for implantable devices such as dextran coated silica beads, stents, or catheters.

The dose of the construct, for example, is dependent on the size of the construct and the purpose for which is it administered. In general, the range is calculated based on the surface area of tissue to be treated. The effective dose of construct may be dependent on the size of the construct and the delivery vehicle/targeting method used and chemical composition of the oligonucleotide but a suitable dose may be determined by the skilled person, for example making use of data from the animal and in vitro test systems indicated above.

The construct, for example, may be administered to the patient systemically for both therapeutic and prophylactic purposes. The construct, for example may be administered by any effective method, as described above, for example, parenterally (eg intravenously, subcutaneously, intramuscularly) or by oral, nasal or other means which permit the construct, for example, to access and circulate in the patient's bloodstream. Construct administered systemically preferably are given in addition to locally administered construct, but also have utility in the absence of local administration.

It is believed that uptake of the nucleic acid and expression of the encoded polypeptide by dendritic cells may be the mechanism of priming of the immune response; however, dendritic cells may not be transfected but are still important since they may pick up expressed peptide from transfected cells in the tissue.

It is preferred if the vaccine, such as DNA vaccine, is administered into the muscle. It is also preferred if the vaccine is administered onto or into the skin.

Conveniently, the nucleic acid vaccine may comprise any suitable nucleic acid delivery means, as noted above. The nucleic acid, preferably DNA, may be naked (ie with substantially no other components to be administered) or it may be delivered in a liposome or as part of a viral vector delivery system.

The nucleic acid vaccine may be administered without adjuvant. The nucleic acid vaccine may also be administered with an adjuvant such as BCG or alum. Other suitable adjuvants include Aquila's QS21 stimulon (Aquila Biotech, Worcester, Mass., USA) which is derived from saponin, mycobacterial extracts and synthetic bacterial cell wall mimics, and proprietory adjuvants such as Ribi's Detox. Quil A, another saponin-derived adjuvant may also be used (Superfos, Denmark). Other adjuvants such as Freund's may also be useful. It is preferred if the nucleic acid vaccine is administered without adjuvant.

Documents and patent applications referred to herein are hereby incorporated by reference.

The invention is now described by reference to the following, non-limiting, figures and examples.

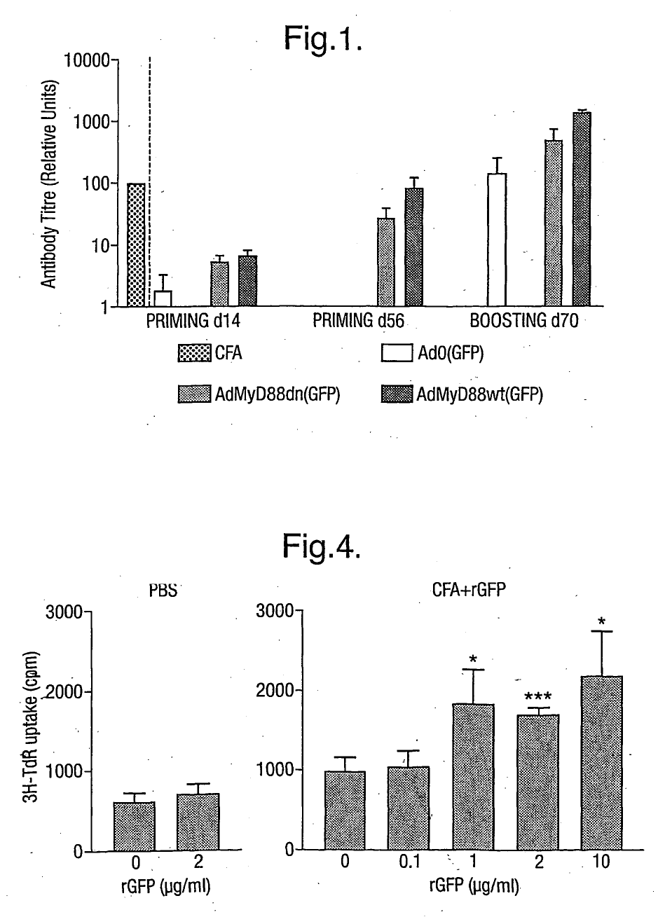

FIG. 1: Immunisation with 10⁷ pfu of AdMyD88dn or AdMyD88wt increases Ad0(GFP)-induced anti-GFP antibody production. Groups of five BALB/c mice (eight to ten weeks old) were immunised subcutaneously with PBS, 20 μg of recombinant GFP emulsified with CFA at a 1:1 ratio, or 10⁷ pfu of recombinant adenoviruses expressing GFP [Ad0(GFP)], GFP with dominant negative MyD88 [AdMyD88dn(GFP)] or GFP with wild-type MyD88 [AdMyD88wt(GFP)]. A total volume of 100 μl/mouse was injected at the base of the tail. After 56 days, mice received a boosting dose of 10⁶ pfu of the same adenovirus that they were with, and antibody levels measured again after another 14 days. At days 14, 56 and 70, mice were tail-bled and serum anti-GFP-specific antibodylevels of each mouse sseparately were assayed in triplicate by ELISA by using a colorimetric assay. Results are expressed as mean relative antibody units (±SME) of 5 mice/group. Relative antibody titers were calculated as described in Williams R. O. et al (1992). The 50% O.D. point of the antibody levels inducted by rGFP and CFA immunisation was used to define “100 relative units”.

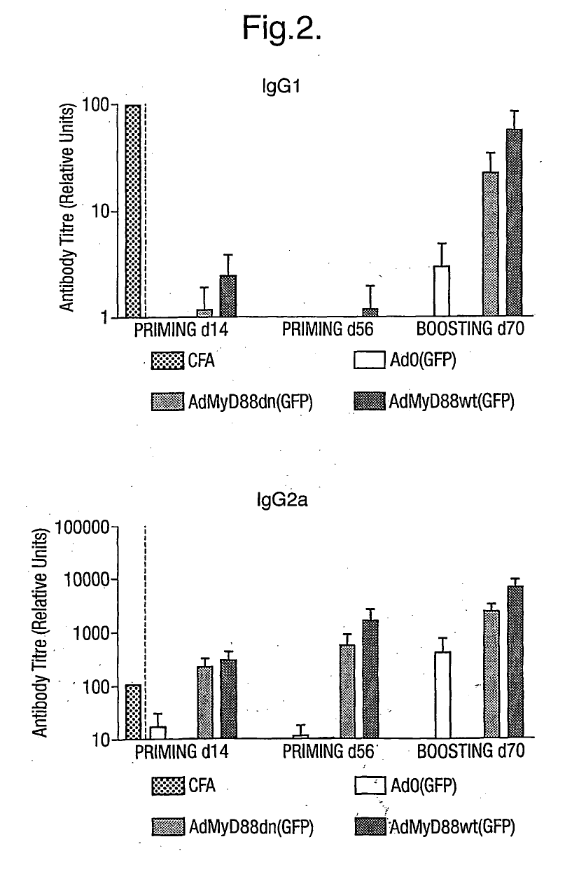

FIG. 2: Immunisation with 10⁷ pfu of AdMyD88dn or AdMyD88wt increases Ad0(GFP)-induced anti-GFP IgG2 antibody production. Immunisation was performed as described in the legend to FIG. 1. At days 14, 56 and 70, mice were tail-bled and serum IgG1 or IgG2a anti-GFP-specific antibody levels of each mouse separately were assayed in triplicate by ELISA using a colorimetric assay. Results are expressed as mean relative antibody units (±SEM) of 5 mice/group. Relative antibody titres were calculated as described in Williams R O et al (1992). The 50% OD point of the antibody levels induced by rGFP and CFA immunisation was used to define “100 relative units”.

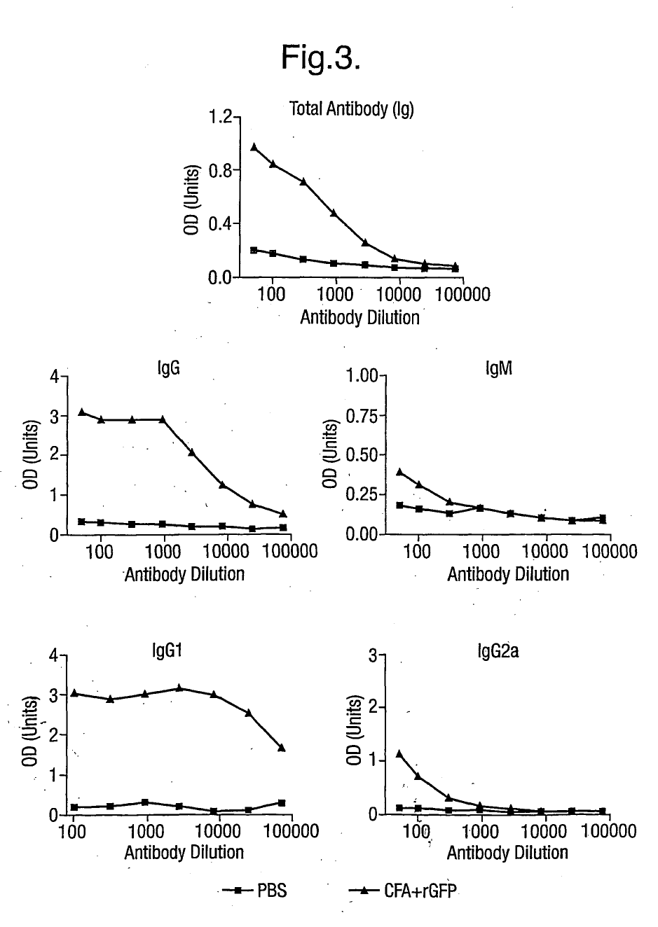

FIG. 3: Immunization of BALB/c mice with recombinant GFP in CFA induces anti-GFP antibody responses. Groups of five BALB/c mice (eight to ten weeks old) were vaccinated by the subcutaneous route with PBS or 20 μg/ml of recombinant GFP emulsified with CFA at a 1:1 ratio. A total volume of 100 μl/mouse was injected at the base of the tail. After 14 days, mice were tail-bled and serum antibody levels of each mouse were assayed separately in triplicates by ELISA by using a colorimetic assay. Total antibody (Ig) as well as IgG, IgM, IgG1 and IgG2a isotypes were measured. Titration curves showing the mean absorbance of each group are shown and are a representative of two independent experiments. The 50% O.D. point of the antibody levels induced by rGFP and CFA immunization was used to define “100 relative arbitrary units” and calculate relative arbitrary units for the other groups (Williams R. O. et al. 1992). Relative arbitrary units were used in FIGS. 1 and 2.

FIG. 4: Immunisation of BALB/c mice for 7 days with recombinant GFP and CFA induces weak lymph node cell proliferation. Groups of five BALB/c mice (eight to ten weeks old) were immunised subcutaneously with PBS or 20 μg of recombinant GFP emulsified with CFA at a 1:1 ratio. A total volume of 100 μl/mouse was inhected at the base of the tail. After 7 days, mice were sacrificed, inguinal lymph nodes excised and cells cultured as single-cell suspensions in the presence or absence of recombinaint GFP. Cells from each mouse were cultured separately in triplicates. Proliferation was measured after 72 h by incorporation of tritiated thymidine. Mean proliferation (±SEM) of 5 mice/group is shown and is representative of two independent experiments. An unpaired students t-test (two-tailed) was used to compare groups with the background proliferation of lymph node cells from PBS control mice (*p<0.05, ***p<0.001).

### EXAMPLE 1

Immunostimulatory Molecules Drive T_{H1} and Not TH2 Response and Can Inhibit T_{H2} Responses

Allergic disease, including asthma, rhinitis, atopic dermatitis, and more severe forms including anaphylaxis, are due to Th2 driven immune respopnses. We have unexpectedly found that immunostimulatory molecules activiting dendritic cells induce responses that are chiefly T_{H1}, even in mouse strains like BALB/c genetically prone to T_{H2} type responses. On boosting, the Th1 response increases. Thus, agents of this type may be useful in reprogramming the immune system away from the allergic phenotype.

In this Example, we have examined the effect of NF-κB activation and activation of APCs in the induction of immune responses in vivo.

Several types of genetic vaccines exist, including viral, bacterial or naked DNA vaccines. Viral or bacterial vectors that invade the cytoplasm of cells are routinely used in experiment protocols of vaccination. These include adenoviruses, vaccinia viruses, Salmonella, Mycobacterium bovis bacillus Calmette-Guerin (BCG) or Listeria monocytogenes, and offer the advantage of introducing antigens directly into the antigen-presenting cells (Panicali D. et al. 1983; Morin J. E. et al. 1987; Dietrich G. et al. 1999). Their disadvantages, however, include the potential to cause disease in humans, especially immunocompromised individuals, and the production of neutralizing antibody responses to the vector, that may render further immunizations ineffective. Naked DNA vectors, on the other hand, consist of plasmid expression vectors that allow repeated immunizations to be effective, and seem to be safer than viral vectors, although plasmid integration into the genome could theoretically mutate or disrupt host genes (Tang D. C. et al. 1992; Ulmer J. B. et al. 1992; Fynan E. F. et al. 1993; Donnelly J. J. et al. 1995; Dittmer U. et al. 1998).

In order to determine whether activation of DC (for example by activation of NFκB) provides adjuvant action, a system of DNA vaccination by using replication-deficient adenoviruses as DNA delivery vehicles may be used. As antigen, it is convenient to use green fluorescent protein (GFP), a jellyfish protein that has been previously found to be immunogenic in animals (Stripecké R. et al. 1999). Vaccine studies performed by others to assess the potential of replication-deficient adenoviruses as vaccine vehicles have used the bacterial protein β-galactosidase (β-gal) as a model antigen. As an experimental host, BALB/c mice, a strain that is genetically skewed to practice T_{H2} responses and is commonly used for vaccine studie, may be used. As a route of immunization, subcutaneous immunization that targets skin DC may be used. Thus, it is possible to examine whether incorporation of a gene considered to activate DCs, for example to induce NFκB, into the same viral vector that encodes the prototype antigen GFP, could enhance the immune response against that antigen, and whether it could skew it to a T_{H1} profile. Comparisons of that with traditional animal adjuvants such as complete Freund's adjuvant (CFA) were also included.

There are several ways of measuring the immune response against a specific antigen. Commonly, antibody responses that are indicative of B cell responses (De Franco A. L. 1987), and lymph node cell proliferation responses that are indicative of T cell responses in animals (Alkan S. S. 1978), are evaluated. The presence of cytotoxic responses is tested by measuring the ability of cytotoxic T lymphocytes to kill target cells, whereas cell-mediated immune responses are examined by measuring the proliferation of lymph node cells and the delayed-type hypersensitivity reaction (DTH). To test whether immunization is protective against allergy, the challenge of the organism with the allergy-causing agent may be performed, and disease progression assessed.

This Example provides evidence that the use of an activator of APCs, for example DCs, for example the incorporation of an NF-κB-activating intracellular signalling molecules into DNA, may be a useful way of enhancing and skewing the immune response towards T_{H1}-type immunity (the type of immunity needed for efficient protection against viruses, various parasites and cancer) and may therefore be useful in increasing the T_{H1}:T_{H2} ratio of an immune response. This may be useful in the treatment of allergy.

A dominant negative mutant of MyD88, for example MyD881pr, is considered to be an activator of APCs, for example DCs. Wild-type MyD88 (MyD88wt) is considered to be an activator of other cell types, for example fibroblasts.

Immunization With Recombinant GFP and Complete Freud's Adjuvant Induces Strong Humoral Responses

A recent study has shown that the jellyfish Aequorea victoria protein GFP induces a strong immune response that results in the lysis of GFP-expressing leukaemic cells in BALB/c mice (Stripecke R. et al. 1999). To confirm the study by Stripecke and colleagues, BALB/c mice were immunized subcutaneously with 20 μg of recombinant GFP emulsified in CFA, and GFP-specific antibody responses were measured 14 days after immunization. A strong antibody response against GFP was detected in immunized animals.

The IgG isotype profile of the GFP-specific antibody response contained high IgG1 and low IgG2a levels (FIG. 3). This suggested that immunization with recombinant GFP in CFA induces mainly T_{H2} responses in BALB/c mice, as IgG2a antibody levels correlate with T_{H1} and IgG1 antibody levels with T_{H2} profiles (Mosmann T. R. and Coffman R. L. 1989).

Immunization With a Replication-Deficient Adenovirus Expressing GFP Induces Only Weak Antibody Responses That Can Be Significantly Enhanced By the Co-Expression of an Activating Gene

Having shown that the prototype antigen GFP induces strong antibody responses in BALB/c mice immunized with recombinant protein and CFA, it was tested whether administration of GFP by replication-deficient adenoviral vectors could also do the same.

A low dose of 10⁶ pfu of test adenovirus vectors produced negligible antibody responses. When mice were immunized for 14 days with a higher titre of 10⁷ pfu of an adenovirus overexpressing GFP [Ad0(GFP)], anti-GFP antibody production was induced. Although this was low compared to that induced by immunization with recombinant GFP and CFA, it was substantially increased with the incorporation of MyD88wt (wildtype) or MyD88dn (dominant negative) into the adenoviral vector expressing GFP [AdMyD88dn(GFP) or AdMyD88wt(GFP)] (FIG. 1).

The antibody levels induced by immunization with Ad0(GFP), AdMyD99dn(GFP) or AdMyD88wt(GFP) consisted mainly of the IgG isotype.

The type of antibody response induced is indicative of the immune response generated. Thus, the production of IgG2a antibody is associated with a T_{H1} profile, whereas the production of IgG1 is associated with a T_{H2} profiles (Mosmann T. R. and Coffman R. L. 1989). In this study, it was found that immunization with Ad0(GFP) induces both IgG2a and IgG1 subtypes, which are still very low when compared to immunization with rGFP and CFA. Immunization with AdMyD88dn(GFP) or AdMyD88wt(GFP), however, induces a strong IgG2a response, whereas an IgG1 response is very low compared with CFA control (FIG. 2). The IgG2a response is at least 10-fold stronger than that induced by recombinant GFP and CFA, suggesting that AdMyD88wt(GFP) or AdMyD88nt(GFP) not only enhances the antibody response against vector-encoded antigen, but also skews the immune response towards a T_{H1} cytokine profile to a much greater extent than that achieved with CFA.

To test whether immunization of BALB/c mice with AdMyD88wt(GFP) or AdMyD88dn induces long-lived antibody responses, I examined the kinetics of antibody production. I found that high anti-GFP-specific antibody levels persisted after 56 days post-immunization (FIG. 1). Similar results were obtained for the IgG2a isotypes, whereas the levels of IgG1 remained very low throughout all this period (FIG. 2). Immunization of BALB/c mice with Ad0(GFP), on the other hand, induced very low levels of antibody production that did not increase further after 56 days FIGS. 1 and 2). Thus, the timecourse of antibody production in our system of adenoviral immunization is different from that observed in other systems of genetic vaccination. Two recent studies examined the immune response against β-galactosidase in BALB/c mice. The first used naked DNA immunization encoding the antigen and found that β-galactosidase-specific IgG2a antibody responses were low 14 days after immunization and increased thereafter (Raz E. et al. 1996). The second employed a replication-deficient adenovirus with similar findings; anti-β-galactosidase IgG levels increased at longer time-points and remained high even after 6 months post-immunization without boosting (Juillard V. et al. 1995).

Overall, these data suggest that in the absence of an activator gene overexpression, a dose of 10⁷ pfu. of Ad0(GFP) is not sufficient to induce antibody responses, and a higher dose of Ad0(GFP) may be needed. If, however, an activator gene such as MyD88wt or MyD88dn gets incorporated into the adenoviral vector, the same dose of 10⁷ pfu of the replication-deficient adenoviral vector is able to generate potent and long-lasting immune responses against a vector-encoded antigen.

Rechallenge of Mice With AdMyD88dn(GFP) or AdMyD88wt(GFP) Boosts IgG2a and T_{H1} Responses

Primary immunization of BALB/c mice with 10⁷ pfu of AdMyD88dn(GFP) or AdMyD88wt(GFP) induces potent IgG2a antibody responses whereas at that titre Ad0(GFP) was not effective. Next, the ability of AdMyD88dn(GFP) or AdMyD88wt(GFP) or Ad0(GFP) to boost the antibody responses was investigated. Thus, 56 days after the primary immunization, BALB/c mice immunized, with Ad0(GFP) or AdMyD88dn(GFP) or AdMyD88wt(GFP) received a boosting dose of 10⁶ pfu of Ad0(GFP) or AdMyD88dn(GFP) or, AdMyD88wt(GFP), respectively, and antibody levels were measured after a further 14 days. It was found that a secondary immunization was capable of boosting antibody responses in both Ad0(GFP)- and AdMyD88dn(GFP) or AdMyD88wt(GFP)-immunized mice (FIGS. 1 and 2). Re-administration of Ad0(GFP) increases Ig, IgG2a and IgG1 isotypes compared to the primary immunization, although the most pronounced increase was observed in IgG2a. Similarly, re-administration of AdMyD88dn(GFP) or AdMyD88wt(GFP) increases Ig and IgG2a antibody levels. These data suggest that a secondary immunization with a 10-fold lower dose of replication-deficient adenoviruses effectively boosts antibody levels and maintains the skew of the immune response towards a T_{H1} profile.

### Discussion

In the past ten years, genetic immunization has emerged as a new approach to vaccine development. Through genetic immunization, the gene encoding a target antigen can be introduced into the cytoplasm of a cell, resulting in effective processing and antigen presentation, and inducing humoral and cell-mediated immune responses in vivo (Tang D. C. et al. 1992; Ulmer J. B. et al. 1992; Fynan E. F. et al. 1993; Donnelly J. J. et al. 1995; Dittmer U. et al. 1998; Panicali D. et al. 1983; Morin J. E. et al. 1987). Several gene transfer methods can be used for that purpose, including retroviral, adenoviral and vaccinia virus gene transfer, or direct injection of naked DNA. They offer significant advantages over alternative immunization strategies, as they are replication-deficient, stable and are relatively easy to prepare. However, and despite encouraging early results, the levels of specific immunity induced by these vectors has not been sufficient to provide long-lived protection against challenge with pathogenic organisms. Thus, vaccination of humans with naked DNA has been disappointing in comparison with the rodent models (Wang R. et al. 1998; Le T. P. et al. 2000; Calarota S. et al. 1998), and the use of attenuated vaccinia viruses has shown little efficacy (Seder R. A. and Hill A. V. S. 2000). This prompted investigators to try to optimize the immunogenicity of genetic vaccines themselves or use priming/boosting immunization strategies with naked DNA and viral vaccines to enhance immunity.

To optimize genetic vaccines in humans, most approaches have focused on improving immunogenicity of vector-encoded antigens. The intrinsic immunogenicity of naked DNA vaccines is mainly due to undermethylated CpG motifs, specific nucleotide sequences of viral or bacterial genes found within the plasmid, that have been shown in human and mice to stimulate the immune system, inducing T_{H1} and cytotoxic CD8⁺ T lymphocyte responses (Cho H. J. et al. 2000; Cowdery J. S. et al. 1996; Klinman D. M. et al. 1996; Sato Y. et al. 1996). To enhance or skew the immune responses generated by DNA vaccination, several groups have introduced various cytokine, chemokine, costimulatory molecules, or combinations of them to the DNA backbone. These studies and their effects on humoral and cellular immune responses have been recently reviewed by Gurunathan and colleagues (table 6.1). Although promising, their efficacy and safety in humans remains questionable.

Live virus vectors, on the other hand, generate stronger cellular immune responses than do DNA vaccines in small animals. But if poxviruses and adenoviruses are used in humans or other animals with pre-existing immunity against the viral vectors, their efficacy dramatically decreases. Pre-existing immunity reduces the expression of the transgene by destroying cells expressing the transgene and by diminishing the ability of the virus to deliver the transgene (Yang Y. et al. 1994; Kuriyama S. et al. 1998). To circumvent this problem, less immunogenic vectors such as adeno-associated virus, lentivirus or gutless adenovirus are being tested. At the same time, different methods of viral delivery look promising (Siemens D. R. 2001).

Recently, a novel strategy involving a heterologous prime-boost immunization has been shown to be helpful. It makes use of naked DNA vaccines for priming and recombinant viral vectors encoding the same foreign antigens for boosting the immune response. This approach has been demonstrated to be effective in several infectious diseases in mice and in primates, leading to substantial enhancement of T_{H1} and cytotoxic T lymphocyte responses (Schneider J. et al. 1998; Schneider J. et al. 1999; Gilbert S. C. et al. 1999). As viral vectors, several poxviruses, such as modified vaccinia virus Ankara (MVA) and fowlpox, as well as replication-defective adenoviruses have this capacity to boost a primed cytotoxic T lymphocyte response substantially (Kent S. J. et al. 1998; Hanke T. et al. 1999; Rothel J. S. et al. 1997). This approach is now under clinical trials in malaria and HIV.

TABLE 6.1
Incorporation of cytokines/chemokines and costimulatory molecules as a
way of enhancing or regulating immunity induced by DNA vaccines
(adapted from Gurunathan S. et al. 2000).

| Cytokine | Antibody | Cellular response | CTL |
|---|---|---|---|
| IL-1 | ↑IgG | ↑proliferation | ↑CTL |
| | ↑IgG2a | ↑IFNγ | |
| IL-2 | ↑IgG | ↑proliferation | ↑CTL |
| | ↑IgG2a | ↑IFNγ | |
| IL-4 | ↑IgG | ↑proliferation | |
| | ↑IgG1 | ↓DTH | |
| | | ↑IL-4 | |
| IL-5 | ↑IgG | ±proliferation | |
| | | ↑IFNγ | |
| IL-7 | ↑IgG2a | ↑IFNγ | |
| | ↑IgG1 | | |
| IL-8 | | ↑Neutrophils | |
| | | ↓DTH | |
| IL-10 | ↑IgG | ↓DTH | |
| | ↓IgG2a | ↓proliferation | |
| IL-12 | ↑IgG2a | ↑DTH | |
| | IgG1? | ↑proliferation | |
| | IgG? | ↑IFNγ | |
| IL-15 | IgG? | ±↑proliferation | ↑CTL |
| IL-18 | ↑IgG | ↑proliferation | ↑CTL |
| TNF | ↑IgG | ↑proliferation | ↑CTL |
| GM-CSF | ↑IgG | ↑proliferation | ↑CTL |
| | ↑IgG2a | ↑IFNγ | |
| | ↑IgG1 | ↑IL-4 | |
| TGF-β | ↑IgG1 | ↓DTH | |
| | | ↓proliferation | |
| | | ↓cytokines | |
| IFNγ | ↑IgG2a | ?Proliferation | ↑CTL |
| | ?IgG? | ↑IFNγ | |
| | | ↓IL-5 | |
| CD80 | | | ↑CTL |
| CD86 | | ↑DTH | ↑CTL |
| | | ↑Proliferation | |
| CD40L | ↑IgG | ↑IFNγ | ↑CTL |
| | ↑IgG2a | | |
| | ↑IgG1 | | |
| CD54 | | ↑proliferation | ↑CTL |
| | | ↑IFNγ | |
| LFA-3 | | ↑proliferation | ↑CTL |
| | | ↑IFNγ | |
| CTLA4 | ↑IgG | ↑proliferation | |
| | ↑IgG1/IgG2a | | |

Expression of an NF-κB-inducer into immature dendritic cells is considered to enhance their antigen-presenting function. It induces the activation of p⁶⁵, relB and p50 NF-κB subunits, and it coordinates the up-regulation of cytokines, chemokines, MHC antigen-presenting and costimulatory molecules. Genetic immunization has been shown to work through the direct or indirect transfection of dendritic cells (Corr M. et al. 1996; Doe B. et al. 1996; Condon C. et al. 1996; Raz E. et al. 1994; Albert M. L. et al. 1998), the most potent antigen-presenting cells. We have devised a model of genetic immunization against a model antigen green fluorescent protein (GFP) by replication-deficient adenoviral vectors to compare humoral and cell-mediated immune responses. For example, adenoviruses expressing GFP alone [Ad0(GFP)], or GFP together with an activator gene, for example NFκB-inducing gene, or MyD88dn or MyD88wt as an adjuvant [AdMyD88dn(GFP) or AdMyD88wt(GFP)] may be compared.

First, we examined whether the jellyfish protein GFP used as a model antigen is immunogenic in BALB/c mice. Administration of recombinant GFP in CFA subcutaneously induces a strong humoral immune response against the antigen that can be measured after 14 days and that consists of high IgG1 but low IgG2a levels. At the same time, immunization with GFP in CFA induces only weak antigen-specific proliferation of lymph node cells.

We investigated whether replication-deficient adenoviruses carrying the GFP gene could also be used to induce an immune response against GFP. We have found that a dose of 10⁷ pfu of recombinant adenovirus was required for detectable antigen-specific responses after intradermal immunization of BALB/c mice, as lower doses were not effective. Thus, administration of Ad0(GFP) induced antigen-specific lymph node cells proliferation but only negligible antibody production. Incorporation of MyD88dn (which may act as an APC activator and/or NFκB inducer) or MyD88wt (which may act as an activator and/or NFκB inducer in other cell types such as fibroblasts) into the adenoviral vector, however, significantly increased both lymph node cell proliferation and anti-GFP antibody production, suggesting that activator/NFκB inducer genes, for example MyD88wt and MyD88dn, have a potent adjuvant effect in the immunogenicity of the vector-encoded antigen. AdMyD88dn(GFP) or AdMyD88wt-induced antibody production consisted mainly of the IgG2a isotype with undetectable levels of IgG1. These findings suggest that although adenoviral immunization favours cell-mediated immune responses when compared to the humoral responses induced by the administration of recombinant protein and adjuvant, incorporation of MyD88dn or MyD88wt into the adenoviral vector significantly enhances that effect and skews the response towards a T_{H1} cytokine profile and cell-mediated immunity. This effect is so strong that a single immunization is sufficient to overcome the genetic predisposition of BALB/c mice to generate T_{H2}-type responses (Heinzel F. P. et al. 1989).

I next examined whether the levels of antibody production induced by AdMyD88wt(GFP) or AdMyD88dn were long-lived and I found that 56 days after immunization, high serum levels of total Ig and IgG2a anti-GFP antibody were still present. These levels remained stable and did not increase or decrease significantly during that period. This is in contrast to the studies of others with naked DNA immunization, where IgG2a antibody levels against a vector-encoded model antigen, β-galactosidase, increased at later time-points (Kaz E. et al. 1996).

Finally, I investigated whether a second administration of recombinant adenoviruses could boost the immune responses and thus provide superior immunity. For that purpose mice received a booster immunization of 10⁶ pfu of recombinant adenovirus 56 days after the priming immunization, a dose which by itself is not capable of providing a useful primary response. I have found that administration of Ad0(GFP) to already AD0(GFP) immunized mice induces high levels of antibody production that correlate with a mixed T_{H1}/T_{H2} response as both IgG2a and IgG1 levels could be measured. Similarly, administration of AdMyD88dn(GFP) to AdMyD88dn(GFP)-immunised mice or administration of AdMyD88wt(GFP) to AdMyD88wt(GFP)-immunised mice further increases the total anti-GFP-specific antibody levels. The response remains skewed to the T_{H1} profile. In summary, these data show that a second administration of replication-deficient adenoviruses can boost the antibody levels against the vector-encoded antigen. But although this induces a mixed Tₕ₁/Tₕ₂ response, incorporation of an activator/inducer or MyD88 gene into the adenoviral vector skews that response to the T_{H1} type. In addition, MyD88wt or MyD88dn increases both total and IgG2a antibody levels, suggesting that it has at the same time a potent adjuvant effect.

This is the first study that makes use of an intracellular signalling molecule as an adjuvant to enhance the immunogenicity of genetic vaccines. It is based on the observation that the expression of MyD88dn in immature DC coordinates the production of cytokines and chemokines, and the up-regulation of MHC antigen-presenting and costimulatory molecules. In vivo, incorporation of MyD88d or MyD88wt into adenoviral DNA vectors leads to enhanced antigen-specific T cell and IgG2a antibody responses, that correlate with a T_{H1}-type of immunity (Mosmann T. R. and Coffman R. L. 1989).

The implications of these findings are very important. First, MyD88wt or MyD88dn or other activator/NFκB inducer genes may be very useful adjuvants for genetic immunization against viral and certain parasitic or bacterial infections, or even cancer vaccines that require strong cell-mediated immune responses. Second, the strong skewing effect induced by MyD88wt or MyD88dn towards T_{H1} immunity indicates that it may be very useful for the treatment of allergy. In various studies, vaccination with allergen in the form of naked plasmid DNA has been shown to stimulate T_{H1}-type allergen-specific immune responses that confer long-lasting protection against allergy (Donnelly J. J. et al. 1997; Roman M. et al. 1997). But although this approach is successful in preventing allergic diseases, the therapy of ongoing conditions has been limited, with the exception of a recent report showing that the incorporation of the IL-18 gene into the vector can successfully reverse airway hyperresponsiveness in mice (Maecker H. T. et al. 2001). The ability of MyD88wt or MyD88dn or other activator/inducer genes to induce strong T_{H1} immune responses make it an attractive way of reprogramming the responses against an allergen. Finally, these data define a novel family of vaccine adjuvants that consist of intracellular signalling molecules involved in the regulation of the immune response. Activation of the immune response in that way may provide a more physiological approach of enhancing immunogenicity by upregulating many functions involved in immunity, compared to the artificial expression of single cytokines or costimulatory molecules that may result in increased toxicity of vaccines and safety concerns.

### References

Albert, M. L., Pearce, S. F., Francisco, L. M., Sauter, B., Roy, P., Silverstein, R. L., and Bhardwaj, N. (1998a). Immature dendritic cells phagocytose apoptotic cells via alphavbeta5 and CD36, and cross-present antigens to cytotoxic T lymphocytes. J Exp Med 188, 1359-68.

Albert, M. L., Sauter, B., and Bhardwaj, N. (1998b). Dendritic cells acquire antigen from apoptotic cells and induce class I-restricted CTLs. Nature 392, 86-9.

Alkan, S. S. (1978). Antigen-induced proliferation assay for mouse T lymphocytes. Response to a monovalent antigen. Eur J Immunol 8, 112-8.

Calarota, S., Bratt, G., Nordlund, S., Hinkula, J., Leandersson, A. C., Sandstrom, E., and Wahren, B. (1998). Cellular cytotoxic response induced by DNA vaccination in HIV-1-infected patients. Lancet 351, 1320-5.

Cho, H. J., Takabayashi, K., Cheng, P. M., Nguyen, M. D., Corr, M., Tuck, S., and Raz, E. (2000). Immunostimulatory DNA-based vaccines induce cytotoxic lymphocyte activity by a T-helper cell-independent mechanism. Nat Biotechnol 18, 509-14.

Condon, C., Watkins, S. C., Celluzzi, C. M., Thompson, K., and Falo, L. D. (1996). DNA-based immunization by in vivo transfection of dendritic cells. Nat Med 2, 1122-8.

Corr, M., Lee, D. J., Carson, D. A., and Tighe, H. (1996). Gene vaccination with naked plasmid DNA: mechanism of CTL priming. J Exp Med 184, 1555-60.

Cowdery, J. S., Chace, J. H., Yi, A. K., and Krieg, A. M. (1996). Bacterial DNA induces NK cells to produce IFN-gamma in vivo and increases the toxicity of lipopolysaccharides. J Immunol 156, 4570-5.

DeFranco, A. L. (1987). Molecular aspects of B-lymphocyte activation. Annu Rev Cell Biol 3, 143-78.

Dietrich, G., Gentschev, I., Hess, J., Ulmer, J. B., Kaufmann, S. H., and Goebel, W. (1999). Delivery of DNA vaccines by attenuated intracellular bacteria. Immunol Today 20, 251-3.

Dittmer, U., Brooks, D. M., and Hasenkrug, K. J. (1998). Characterization of a live-attenuated retroviral vaccine demonstrates protection via immune mechanisms. J Virol 72, 6554-8.

Doe, B., Selby, M., Barnett, S., Baenziger, J., and Walker, C. M. (1996). Induction of cytotoxic T lymphocytes by intramuscular immunization with plasmid DNA is facilitated by bone marrow-derived cells. Proc Natl Acad Sci USA 93, 8578-83.

Donnelly, J. J., Friedman, A., Martinez, D., Montgomery, D. L., Shiver, J. W., Motzel, S. L., Ulmer, J. B., and Liu, M. A. (1995). Preclinical efficacy of a prototype DNA vaccine: enhanced protection against antigenic drift in influenza virus. Nat Med 1, 583-7.

Donnelly, J. J., Ulmer, J. B., Shiver, J. W., and Liu, M. A. (1997). DNA vaccines. Annu Rev Immunol 15, 617-48.

Fynan, E. F., Webster, R. G., Fuller, D. H., Haynes, J. R., Santoro, J. C., and Robinson, H. L. (1993). DNA vaccines: protective immunizations by parenteral, mucosal, and gene-gun inoculations. Proc Natl Acad Sci USA 90, 11478-82.

Gilbert, S. C., Schneider, J., Plebanski, M., Hannan, C. M., Blanchard, T. J., Smith, G. L., and Hill, A. V. (1999). Ty virus-like particles, DNA vaccines and Modified Vaccinia Virus Ankara; comparisons and combinations. Biol Chem 380, 299-303.

Gurunathan S., Wu, C. Y., Freidag, B. L., and Seder, R. A. (2000). DNA vaccines: a key for inducing long-term cellular immunity. Curr Opin Immunol 12, 442-7.

Gurunathan, S., Klinman, D. M., and Seder, R. A. (2000). DNA vaccines: immunology, application, and optimization*. Annu Rev Immunol 18, 927-74.

Hanke, T., Samuel, R. V., Blanchard, T. J., Neumann, V. C., Allen, T. M., Boyson, J. E., Sharpe, S. A., Cook, N., Smith, G. L., Watkins, D. I., Cranage, M. P., and McMichael, A. J. (1999). Effective induction of simian immunodeficiency virus-specific cytotoxic T lymphocytes in macaques by using a multiepitope gene and DNA prime-modified vaccinia virus Ankara boost vaccination regimen. J Virol 73, 7524-32.

Heinzel, F. P., Sadick, M. D., Holaday, B. J., Coffman, R. L., and Locksley, R. M. (1989). Reciprocal expression of interferon gamma or interleukin 4 during the resolution or progression of murine leishmaniasis. Evidence for expansion of distinct helper T cell subsets. J Exp Med 169, 59-72.

Juillard, V., Villefroy, P., Godfrin, D., Parivani, A., Venet, A., Guillet, J. G. (1995). Long-term humoral and cellular immunity induced by a single immunization with replication-defective recombinant adenovirus vector. Eur. J Immunol 25, 3467-73.

Kent, S. J., Zhao, A., Best, S. J., Chandler, J. D., Boyle, D. B., and Ramshaw, I. A. (1998). Enhanced T-cell immunogenicity and protective efficacy of a human immunodeficiency virus type 1 vaccine regimen consisting of consecutive priming with DNA and boosting with recombinant fowlpox virus. J Virol 72, 10180-8.

Klinman, D. M., Yi, A. K., Beaucage, S. L., Conover, J., and Krieg, A. M. (1996). CpG motifs present in bacteria DNA rapidly induce lymphocytes to secrete interleukin 6, interleukin 12, and interferon gamma. Proc Natl Acad Sci USA 93, 2879-83.

Kuriyama, S., Tominaga, K., Kikukawa, M., Nakatani, T., Tsujinoue, H., Yamazaki, M., Nagao, S., Toyokawa, Y., Mitoro, A., and Fukui, H. (1998). Inhibitory effects of human sera on adenovirus-mediated gene transfer into rat liver. Anticancer Res 18, 2345-51.

Le, T. P., Coonan, K. M., Hedstrom, R. C., Charoenvit, Y., Sedegah, M., Epstein, J. E., Kumar, S., Wang, R., Doolan, D. L., Maguire, J. D., Parker, S. E., Hobart, P., Norman, J., and Hoffman, S. L. (2000). Safety, tolerability and humoral immune responses after intramuscular administration of a malaria DNA vaccine to healthy adult volunteers. Vaccine 18, 1893-901.

Maecker, H. T., Hansen, G., Walter, D. M., DeKruyff R. H., Levy, S., and Umetsu, D. T. (2001). Vaccination with allergen-IL-18 fusion DNA protects against, and reverses established, airway hyperreactivity in a murine asthma model. J Immunol 166, 959-65.

Morin, J. E., Lubeck, M. D., Barton, J. E., Conley, A. J., Davis, A. R., and Hung, P. P. (1987). Recombinant adenovirus induces antibody response to hepatitis B virus surface antigen in hamsters. Proc Natl Acad Sci USA 84, 4626-30.

Mosmann, T. R., and Coffman, R. L. (1989). TH1 and TH2 cells: different patterns of lymphokine secretion lead to different functional properties. Annu Rev Immunol 7, 145-73.

Mosmann, T. R., and Sad, S. (1996). The expanding universe of T-cell subsets: Th1, Th2 and more. Immunol Today 17, 138-46.

Panicali, D., Davis, S. W., Weinberg, R. L., and Paoletti, E. (1983). Construction of live vaccines by using genetically engineered poxviruses: biological activity of recombinant vaccinia virus expressing influenza virus hemagglutinin. Proc Natl Acad Sci USA 80, 5364-8.

Raz, E., Carson, D. A., Parker, S. E., Parr, T. B., Abai, A. M., Aichinger, G., Gromkowski, S. H., Singh, M., Lew, D., Yankauckas, M. A., and et al. (1994). Intradermal gene immunization: the possible role of DNA uptake in the induction of cellular immunity to viruses. Proc Natl Acad Sci USA 91, 9519-23.

Raz, E., Tighe, H., Sato, Y., Corr, M., Dudler, J. A., Roman, M., Swain, S. L., Spiegelberg, H. L., and Carson, D. A. (1996). Preferential induction of a Th1 immune response and inhibition of specific IgE antibody formation by plasmid DNA immunization. Proc Natl Acad Sci USA 93, 5141-5.

Roman, M., Martin-Orozco, E., Goodman, J. S., Nguyen, M. D., Sato, Y., Ronaghy, A., Kornbluth, R. S., Richman, D. D., Carson, D. A., and Raz, E. (1997). Immunostimulatory DNA sequences function as T helper-1-promoting adjuvants. Nat Med 3, 849-54.

Rothel, J. S., Boyle, D. B., Both, G. W., Pye, A. D., Waterkeyn, J. G., Wood, P. R., and Lightowlers, M. W. (1997). Sequential nucleic acid and recombinant adenovirus vaccination induces host-protective immune responses against Taenia ovis infection in sheep. Parasite Immunol 19, 221-7.

Sato, Y., Roman, M., Tighe, H., Lee, D., Corr, M., Nguyen, M. D., Silverman, G. J., Lotz, M., Carson, D. A., and Raz, E. (1996). Immunostimulatory DNA sequences necessary for effective intradermal gene immunization. Science 273, 352-4.

Schneider, J., Gilbert S. C., Blanchard, T. J., Hanke, T., Robson, K. J., Hannan, C. M., Becker, M., Sinden, R., Smith, G. L., and Hill, A. V. (1998). Enhanced immunogenicity for CD8+ T cell induction and complete protective efficacy of malaria DNA vaccination by boosting with modified vaccinia virus Ankara. Nat Med 4, 397-402.

Schneider, J., Gilbert, S. C., Hannan, C. M., Degano, P., Prieur, E., Sheu, E. G., Plebanski, M., and Hill, A. V. (1999). Induction of CD8+ T cells using heterologous prime-boost immunisation strategies. Immunol Rev 170, 29-38.

Seder, R. A., and Gurunathan, S. (1999). DNA vaccines—designer vaccines for the 21st century. N Engl J Med 341, 277-8.

Seder, R. A., and Hill, A. V. (2000). Vaccines against intracellular infections requiring cellular immunity. Nature 406, 793-8.

Siemens, D. R., Elzey, B. D., Lubaroff, D. M., Bohlken, C., Jensen, R. J., Swanson, A. K., and Ratliff, T. L. (2001). Cutting edge: restoration of the ability to generate CTL in mice immune to adenovirus by delivery of virus in a collagen-based matrix. J Immunol 166, 731-5.

Stripecke, R., Carmen Villacres, M., Skelton, D., Satake, N., Halene, S., and Kohn, D. (1999). Immune response to green fluorescent protein: implications for gene therapy. Gene Ther 6, 1305-12.

Tang, D. C., DeVit, M., and Johnston, S. A. (1992). Genetic immunization is a simple method for eliciting an immune response. Nature 356, 152-4.

Ulmer, J. B., Donnelly, J. J., Parker, S. E., Rhodes, G. H., Felgner, P. L., Dwarki, V. J., Gromkowski, S. H., Deck, R. R., DeWitt, C. M., Friedman, A., and et al. (1993). Heterologous protection against influenza by injection of DNA encoding a viral protein. Science 259, 1745-9.

Wang, R., Doolan, D. L., Le. T. P., Hedstrom, R. C., Coonan, K. M., Charoenvit, Y., Jones, T. R., Hobart, P., Margalith, M., Ng, J., Weiss, W. R., Sedegah, M., de Taisne, C., Norman, J. A., and Hoffman, S. L. (1998). Induction of antigen-specific cytotoxic T lymphocytes in humans by a malaria DNA vaccine. Science 282, 476-80.

Yang, Y., Nunes, F. A., Berencsi, K., Furth, E. E., Gonczol, E., and Wilson, J. M. (1994). Cellular immunity to viral antigens limits E1-deleted adenoviruses for gene therapy. Proc Natl Acad Sci USA 91, 4407-11.

### Numbered References

1. Rock et al (1998) PNAS 95, 588-593

2. O'Neil & Dinarello (2000) Immunol Today 21, 206-209

3. Poltorak et al (1998) Science 282, 2085-2088

4. Underhill et al (1999) Nature 401, 811-815

5. Burns et al (1998) J Biol Chem 273, 12203-12209

6. Kawai et al (1999) Immunity 11, 115-122

7. Takeuchi et al (2000) Int Immunol 163, 978-984

8. Du et al (2000) Eur Cytokine Netw 11, 362-371

### EXAMPLE 2

Dendritic Cell Culture

Exemplary Dendritic Cell Culture From Normal Volunteers

CD14⁺ peripheral blood monocytes are adhered to tissue culture flasks and cultured in the presence of 1% AB serum, GM-CSF (400 ng/ml) and IL-4 (400 IU/ml) for 7 days. This yields cells with the morphology of DC and a mean of 49% with the CD1a⁺ marker which is indicative of the immature form of the DC capable of taking up and presenting antigen. These cells are then matured to CD83⁺ cells by the addition of TNFα (15 ng/ml), which enables the DC to present antigen to cytotoxic T-cells. 7% of the cells become CD83⁺ within 1 day, but 3 days at least are required for maximum effect. It is possible that monocyte conditioned medium could replace the 1% AB serum but this is probably not desirable.

### EXAMPLE 3

Treatment of Patients With Allergy

Allergic diseases such as asthma, atopic dermatitis, hayfever are driven in large part by Th2 cytokine dependent antibody responses. The most critical Th2 cytokines are IL-4 and IL-5, and the most important antibody response is IgE.

The therapy of allergic disease is currently chiefly symptomatic, with corticosteroids most widely used. However, this has no impact on the under lying abnormal immunology. The invention provides means of downregulating the Th2 type antibody response while upregulating the Th1. This would have the effect of switching off and diluting out the Th2 dependent antibodies which induce the allergic response.

A cDNA construct encoding both the allergen and the sequence activating the Th1 response/inhibitor of Th2 may be used. The latter molecules comes from one of the family of APC activators or NFκB inducing entities, e.g. MyD88 wild type or MyD88 dominant negative, or NIK (NFκB-inducing kinase).

This cDNA construct would be injected repeatedly either intradermally, s/c or i.m. The doses of the construct would be titrated to reach a good Th1 response.

The cDNA construct could be administered as a plasmid, (‘naked DNA’) or as virus. In mice adenovirus is effective, and other viruses such as modified vaccinia or adeno-associated virus are considered likely to be just as effective.

The linkage of the NFκB inducing signal, which promotes Th1 responses and inhibits Th2 to the allergen is convenient, but may not be necessary. An alternative approach is to administer the NFκB inducing stimulus and the allergen separately; yet another is to just administer the NFκB inducing stimulus, and not to administer the allergen, which the individual is exposed to spontaneously by environmental exposure.

As well as administering allergen together with NFκB inducing stimulus, fragments of allergen could be used, as this may avoid augmenting the allergic B cell response, while still modulating the T cell response.

It would be possible to use fragments (peptides) or protein, and to co-administer an NFκB inducing DNA sequence as a plasmid or virus. An NFκB inducing protein would also produce the desired effect.

### Patient Groups

All patients with an allergic disease may be treated. It is preferred that the allergen to which the patient has an allergic reaction is defined e.g. cat allergy, house dust mite, peanuts, wasp and bee venom, pollens, etc, but this may not be essential as environmental exposure to the allergen may be sufficient. Methods by which the allergen to which a patient reacts may be identified are well known to those skilled in the art, as are allergenic molecules to which allergic responses are common.

The method may be useful with patients with hay fever, asthma, allergic dermatitis or other allergic conditions.

### When to Vaccinate

Vaccination may be performed at any stage, like all immunizations, best not performed when there is any intercurrent disease.

Vaccination of asymptomatic children or adults may be desirable, for example with NFκB inducing DNA, or DNA in a virus, plus or minus allergen, to prevent the induction of allergic responses. This may be useful, for example, when there is a family history of allergy or atopy, or when occupational exposure to an allergen (for example latex) is anticipated.

1. A method of increasing the T_{H1}:T_{H2} ratio of an immune response, comprising the step of supplying to an antigen presenting cell (APC) such as a dendritic cell (DC) or precursor cell thereof, an intracellular activator of APC, such as DC, function.

2. A method according to claim 1 which is carried out in a mammal, such as a human.

3. A method according to claim 2 wherein said mammal is in need of an increase in the T_{H1}:T_{H2} ratio of an immune response.

4. A method according to claim 2 wherein said mammal has or is at risk of allergy.

5. A method according to claim 1 wherein said intracellular activator is an intracellular inducer of NFκB.

6-13. (canceled)

14. A method according to claim 1 wherein said intracellular activator is a dominant negative mutant of Myd88 or a polynucleotide encoding a dominant negative mutant of Myd88.

15. A method according to claim 1 wherein said intracellular activator is Myd88 or a polynucleotide encoding Myd88.

16. A method according to claim 1 wherein said intracellular activator is NFκB, a TRAF (including a TRAF 2, 3, 4, 5 and 6,), TRADD, NIK, IKK1, IKK2, IKKγ, TAK1, PKR, NAK, MEKK, p65/relA, c-rel, rel B, p38MAK, p54JNK, p42/44Erk, a MEK (including MEK 1, 2, 3, 4, 5, 6 and 7,) or a MEKK (including MEKK 1, 2 and 3).

17-18. (canceled)

19. The method of claim 14 wherein the dominant negative mutant is MyD881pr.

20-21. (canceled)

22. The method of claim 1 wherein the patient or cell is, has or will be supplied with an allergen.

23. The method of claim 1 wherein the activator is expressed in the cell or patient.

24. The method of claim 23 wherein the patient or cell is administered a polynucleotide capable of expressing the activator in the cell or patient.

25. The method of claim 24 wherein the polynucleotide is an adenovirus vector.

26. A recombinant polynucleotide comprising (1) a portion (modulating portion) encoding an activator as defined in claim 1 and (2) a portion encoding an allergen.

27. A kit of parts, composition or a chimaeric molecule comprising (1) a portion (modulating portion) comprising or encoding an activator as defined in claim 1 and (2) a portion comprising or encoding an allergen.

28. The recombinant polynucleotide of claim 26 wherein the allergen is associated with asthma, rhinitis, atopic dermatitis or hayfever.

29. A method for increasing the T_{H1}:T_{H2} ratio of an immune response in a patient, or for treating a patient with or at risk of allergy, comprising the steps of (1) obtaining antigen presenting cells or precursors, thereof, preferably dendritic cells or precursors thereof, from the patient; (2) contacting said antigen presenting cells with an activator as defined in claim 1 or polynucleotide encoding same and optionally allergen to which modulation of the immune response is required, ex vivo; and (3) reintroducing the so treated antigen presenting cells into the patient.

30. A vaccine effective against an allergy, comprising an effective amount of an activator as defined in claim 1 or polynucleotide encoding same.

31. The vaccine of claim 30 further comprising an allergen or polynucleotide encoding an allergen.

32. The vaccine of claim 30 wherein the vaccine is a nucleic acid vaccine.

33. A pharmaceutical composition comprising a composition or chimaeric molecule as defined in claim 27, and a pharmaceutically acceptable carrier.

34-37. (canceled)

38. The method of claim 22 wherein the allergen is expressed in the cell or patient.

39. The method of claim 38 wherein the patient or cell is administered a polynucleotide capable of expressing the activator in the cell or patient.

40. The method of claim 39 wherein the polynucleotide is administered in an adenovirus vector.

41. The kit of parts, composition or chimaeric molecule of claim 27 wherein the allergen is associated with asthma, rhinitis, atopic dermatitis or hayfever.

42. The method of claim 22 wherein the allergen is associated with asthma, rhinitis, atopic dermatitis or hayfever.

43. A method for increasing the T_{H1}:T_{H2} ratio of an immune response in a patient, or for treating a patient with or at risk of allergy, comprising the steps of (1) obtaining antigen presenting cells or precursors thereof, preferably dendritic cells or precursors thereof, from the patient; (2) contacting said antigen presenting cells with a polynucleotide as defined in claim 26, ex vivo; and (3) reintroducing the so treated antigen presenting cells into the patient.

44. A method for increasing the T_{H1}:T_{H2} ratio of an immune response in a patient, or for treating a patient with or at risk of allergy, comprising the steps of (1) obtaining antigen presenting cells or precursors thereof, preferably dendritic cells or precursors thereof, from the patient; (2) contacting said antigen presenting cells with a chimaeric molecule as defined in claim 27, ex vivo; and (3) reintroducing the so treated antigen presenting cells into the patient.

45. A pharmaceutical composition comprising a polynucleotide as defined in claim 26, and a pharmaceutically acceptable a carrier.

46. A pharmaceutical composition comprising a vaccine as defined in claim 30, and a pharmaceutically acceptable carrier.
